(19) United States
(12) Patent Application Publication
(10) Pub. No.: US 2005/0255536 A1
(43) Pub. Date: Nov. 17, 2005

(54) Method of selectively measuring triglycerides

(75) Inventors: Masahiko Okada, Niigata-shi (JP); Tomohiro Saito, Sagamihara-shi (JP); Hajime Yoshimura, Sagamihara-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: SHINO-TEST CORPORATION, Tokyo (JP); Masahiko OKADA, Niigata (JP)

(21) Appl. No.: 10/516,291

(22) PCT Filed: Jun. 4, 2003
(86) PCT No.: PCT/JP03/07066

§ 371 Date: Dec. 8, 2004

(30) Foreign Application Priority Data
Jun. 10, 2002 (JP) 2002-168738

(51) Int. Cl. C12Q 1/44

(52) U.S. Cl. 435/19

(57) ABSTRACT

The present invention relates to a reagent for selective measurement of triglycerides contained in very low density lipoprotein and intermediate density lipoprotein or in very low density lipoprotein in a test sample, including a first reagent that contains a first selective reaction promoter, which is an ether or ester compound of a polyoxyalkylene capable of reacting lipoprotein lipase selectively with triglycerides contained in low density lipoprotein and high density lipoprotein; lipoprotein lipase; enzymes which catalyze a series of reactions leading to the generation of hydrogen peroxide or a reduced coenzyme from glycerol; and an enzyme which catalyzes a reaction leading to the conversion of hydrogen peroxide or a reduced coenzyme into another substance, and a second reagent that contains a second selective reaction promoter, which is capable of reacting lipoprotein lipase selectively with triglycerides contained in very low density lipoprotein, intermediate density lipoprotein, low density lipoprotein and high density lipoprotein and to a method for selective measurement of triglycerides contained in very low density lipoprotein and intermediate density lipoprotein or in very low density lipoprotein in a test sample which uses the above reagent.

### TECHNICAL FIELD

The present invention relates to a method and reagent for selective measurement of triglycerides contained in very low density lipoprotein and intermediate density lipoprotein or in very low density lipoprotein which is important for clinical diagnosis of arteriosclerosis.

The invention is significant in the fields of chemistry, life science, medical treatment and the like, and particularly in the field of laboratory tests.

### BACKGROUND ART

Cholesterol and triglycerides are essential nutrients for living organisms. They present in blood in such a form that they are packed in an amphipathic “shell” (as lipoproteins) because they are difficult to dissolve in water.

There are several classes of lipoproteins: chylomicron, very low density lipoprotein (VLDL), intermediate density lipoprotein (IDL), low density lipoprotein (LDL) and high density lipoprotein (HDL), which constitute a complex metabolic system.

These lipoproteins each contain cholesterol and triglycerides, and very low density lipoprotein and intermediate density lipoprotein are composed primarily of triglycerides and greatly involved in the development of arteriosclerosis. It is therefore useful to separately measure triglycerides in VLDL and IDL.

Large-scale follow-up surveys of factors involved in the development of arteriosclerosis have demonstrated that LDL cholesterol and the total amount of serum triglycerides (hereinafter, referred to as “total triglycerides”) promote the development of arteriosclerosis, while HDL cholesterol inhibits the development of arteriosclerosis.

Triglycerides constitute a very small part of LDL and HDL, but a large part of chylomicron, VLDL and IDL.

And triglycerides contained in chylomicron have been found not to be a risk factor for arteriosclerosis.

There have been established and widely used several methods which measure total triglycerides contained in lipoproteins without separating lipoproteins according to class (Henry, J. B., Clinical Diagnosis and Management by Laboratory Method, Philadelphia: W. B. Sauders, pp. 189-204).

These methods involves degrading triglyceride present in serum into glycerol by lipoprotein lipase, subsequently converting it into glycerol-3-phosphate by glycerol kinase, and further converting it into dihydroxyacetone-3-phosphate by glycerol-3-phosphate oxidase, followed by calorimetric assay of the generated hydrogen peroxide using a peroxidase system (Trinder reaction system).

There is another method which measures total triglycerides in lipoproteins by generating NADH (a reduced coenzyme) by the action of glycerol-3-phosphate dehydrogenase, instead of glycerol-3-phosphate oxidase, and measuring the NADH.

These methods are widely called enzymatic assays.

There have also been known methods which measure cholesterol contained in LDL or HDL by allowing a particular surfactant and additive to selectively act on LDL or HDL (e.g. JP Patent Publication (Kokai) No. 9-313200, JP Patent Publication (Kokai) No. 9-285298), and such methods have been widely used for the purpose of laboratory tests etc.

As a method for selective measurement of triglycerides in VLDL and/or IDL, however, there has been only one method, ultracentrifugation, whose operating procedures are complicated and there has been neither method nor reagent for selective measurement of triglycerides in VLDL and/or IDL which is readily performable.

However, Dr. Okada, one of the inventors of this invention, has resolved the above described problem and accomplished a method and reagent for selective measurement of triglycerides contained in VLDL and/or IDL (WO 00/60112).

### DISCLOSURE OF THE INVENTION

Accordingly, the object of this invention is to establish a method and reagent which enables the selective measurement of triglycerides contained in very low density lipoprotein (VLDL) and intermediate density lipoprotein (IDL) or in very low density lipoprotein (VLDL) in a test sample while ensuring ease of operation and good accuracy.

More specifically, the object of this invention is to establish a more readily and accurately performable method and reagent for selective measurement of triglycerides contained in VLDL and IDL or in VLDL in a test sample which does not require any complicated operating procedures, such as centrifugation with an ultracentrifuge, and is applicable to automatic analyzers generally in use.

After directing tremendous research effort toward the solution of the above described problem, the inventors of this invention have found that selecting selective reaction promoters from among those described in WO 00/60112 makes more readily and accurately performable the selective measurement of triglycerides contained in VLDL and IDL or in VLDL in a test sample and finally accomplished this invention.

This invention embraces the following:

(1) A method for selective measurement of triglycerides contained in very low density lipoprotein and intermediate density lipoprotein or in very low density lipoprotein in a test sample, comprising the following two steps:

a first step that comprises

1′. exposing and reacting the test sample to and with lipoprotein lipase and some other enzymes, which catalyze a series of reactions leading to the generation of hydrogen peroxide or a reduced coenzyme from glycerol, in the presence of a first selective reaction promoter, which is an ether or ester compound of a polyoxyalkylene capable of reacting lipoprotein lipase selectively with triglycerides contained in low density lipoprotein and high density lipoprotein, to generate hydrogen peroxide or a reduced coenzyme from the triglycerides contained in the low density lipoprotein and the high density lipoprotein in the test sample,

2′. reacting the hydrogen peroxide or reduced coenzyme generated by the reaction 1′ with an enzyme which catalyzes a reaction leading to the conversion of hydrogen peroxide or a reduced coenzyme into another substance, and

3′. eliminating the triglycerides contained in the low density lipoprotein. and the high density lipoprotein by the reactions 1′ and 2′, and

a second step that comprises

1′. subsequently, after the first step, reacting the test sample with lipoprotein lipase and some other enzymes, which catalyze a series of reactions leading to the generation of hydrogen peroxide or a reduced coenzyme from glycerol, in the presence of a second selective reaction promoter, which is capable of reacting lipoprotein lipase selectively with triglycerides contained in very low density lipoprotein, intermediate density lipoprotein, low density lipoprotein and high density lipoprotein, to generate hydrogen peroxide or a reduced coenzyme from the triglycerides contained in the very low density lipoprotein and the intermediate density lipoprotein or in the very low density lipoprotein; and

2′. measuring the hydrogen peroxide or reduced coenzyme generated by the reaction 1′.

(2) The method according to the above description (1), wherein the second selective reaction promoter is an ether or ester compound of a polyoxyalkylene.

(3) The method according to the above description (2), wherein m/n ratio is in the range of 1.1 to 1.2 where m is the average mole number of the added polyoxyalkylene in its ether or ester compound which is used as the first selective reaction promoter and n is the average mole number of the added polyoxyalkylene in its ether or ester compound which is used as the second selective reaction promoter.

(4) The method according to the above description (3), wherein m is in the range of 7.7 to 18 and n is in the range of 7 to 15.

(5) The method according to the above description (3), wherein m is in the range of 11 to 12and n is 10.

(6) The method according to any one of the above descriptions (1) to (5), wherein the ether or ester compound of a polyoxyalkylene which is used as the first selective reaction promoter is at least one selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

(7) The method according to any one of the above descriptions (1) to (6), wherein the second selective reaction promoter is at least one ether or ester compound of a polyoxyalkylene selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

(8) The method according to any one of the above descriptions (1) to (7), wherein the polyoxyalkylene is polyoxyethylene.

(9) The method according to any one of the above descriptions (1) to (8), wherein the first selective reaction promoter is polyoxyethylene nonylphenyl ether in which the average mole number of added polyoxyethylene m is in the range of 11 to 12 and the second selective reaction promoter is polyoxyethylene nonylphenyl ether in which the average mole number of added polyoxyethylene n is 10.

(10) The method according to any one of the above descriptions (1) to (9), wherein the first step and/or the second step is carried out in the presence of a reaction assistant.

(11) The method according to the above descriptions (10), wherein the reaction assistant is a polysaccharide or derivative thereof, a polyanion, a halogen ion, a metal ion, or lectin.

(12) The method according to any one of the above descriptions (1) to (11), wherein the activity of the lipoprotein lipase being present in the first step depends on the concentration of a surfactant, while that of the lipoprotein lipase being present in the second step hardly depends on the concentration of a surfactant.

(13) A reagent for selective measurement of triglycerides contained in very low density lipoprotein and intermediate density lipoprotein or in very low density lipoprotein in a test sample, including

a first reagent that comprises: a first selective reaction promoter, which is an ether or ester compound of a polyoxyalkylene capable of reacting lipoprotein lipase selectively with triglycerides contained in low density lipoprotein and high density lipoprotein; lipoprotein lipase; enzymes which catalyze a series of reactions leading to the generation of hydrogen peroxide or a reduced coenzyme from glycerol; and an enzyme which catalyzes the reaction leading to the conversion of hydrogen peroxide or a reduced coenzyme into another substance, and

a second reagent that comprises a second selective reaction promoter, which is capable of reacting lipoprotein lipase selectively with triglycerides contained in very low density lipoprotein, intermediate density lipoprotein, low density lipoprotein and high density lipoprotein.

(14) The reagent according to the above description (13), wherein the first reagent and/or the second reagent further comprises a substance which is involved in a reaction leading to the derivation of some signal from hydrogen peroxide or a reduced coenzyme.

(15) The reagent according to the above description (13) or (14), wherein the second selective reaction promoter is an ether or ester compound of a polyoxyalkylene.

(16) The reagent according to the above description (15), wherein m/n ratio is in the range of 1.1 to 1.2 where m is the average mole number of the added polyoxyalkylene in its ether or ester compound which is used as the first selective reaction promoter and n is the average mole number of the added polyoxyalkylene in its ether or ester compound which is used as the second selective reaction promoter.

(17) The reagent according to the above description (16), wherein m is in the range of 7.7 to 18 and n is in the range of 7 to 15.

(18) The reagent according to the above description (16), wherein m is in the range of 11 to 12 and n is 10.

(19) The reagent according to any one of the above descriptions (13) to (18), wherein the ether or ester compound of a polyoxyalkylene used as the first selective reaction promoter is at least one selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

(20) The reagent according to any one of the above descriptions (13) to (19), wherein the second selective reaction promoter is at least one ether or ester compound of a polyoxyalkylene selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

(21) The reagent according to any one of the above descriptions (13) to (20), wherein the polyoxyalkylene is polyoxyethylene.

(22) The reagent according to any one of the above descriptions (13) to (21), wherein the first selective reaction promoter is polyoxyethylene nonylphenyl ether in which the average mole number of added polyoxyethylene m is in the range of 11 to 12 and the second selective reaction promoter is polyoxyethylene nonylphenyl ether in which the average mole number of added polyoxyethylene n is 10.

(23) The reagent according to any one of the above descriptions (13) to (22), wherein the first reagent and/or the second reagent further comprises a reaction assistant.

(24) The reagent according to the above descriptions (23), wherein the reaction assistant is a polysaccharide or derivative thereof, a polyanion, a halogen ion, a metal ion, or lectin.

(25) The reagent according to any one of the above descriptions (13) to (24), wherein the activity of the lipoprotein lipase contained in the first reagent depends on the concentration of a surfactant, while that of the lipoprotein lipase contained in the second reagent hardly depends on the concentration of a surfactant.

In the following this invention will be described in detail.

I. Measurement Method

I-1: General Introduction of Measurement Method

The method for selective measurement of triglycerides contained in very low density lipoprotein and intermediate density lipoprotein or in very low density lipoprotein in a test sample in accordance with this invention is carried out in the following first and second steps.

First Step:

1′. Expose and react a test sample to and with lipoprotein lipase and some other enzymes, which catalyze a series of reactions leading to the generation of hydrogen peroxide or a reduced coenzyme from glycerol, in the presence of a first selective reaction promoter, which is an ether or ester compound of a polyoxyalkylene capable of reacting lipoprotein lipase selectively with triglycerides contained in low density lipoprotein and high density lipoprotein, to generate hydrogen peroxide or a reduced coenzyme from the triglycerides contained in the low density lipoprotein and the high density lipoprotein in the test sample.

2′. React the hydrogen peroxide or reduced coenzyme generated by the reaction of the above description 1′ with an enzyme which catalyzes the reaction leading to the conversion of hydrogen peroxide or a reduced coenzyme into another substance.

3′. Eliminate the triglycerides contained in the low density lipoprotein and the high density lipoprotein by the above reactions 1′ and 2′.

Second Step:

1′. Subsequently after the first step, react the test sample with lipoprotein lipase and some other enzymes, which catalyze a series of reactions leading to the generation of hydrogen peroxide or a reduced coenzyme from glycerol, in the presence of a second selective reaction promoter, which is capable of reacting lipoprotein lipase selectively with triglycerides contained in very low density lipoprotein, intermediate density lipoprotein, low density lipoprotein and high density lipoprotein, to generate hydrogen peroxide or a reduced coenzyme from the triglycerides contained in the very low density lipoprotein and the intermediate density lipoprotein or in the very low density lipoprotein.

2′. Measure the hydrogen peroxide or reduced coenzyme generated by the above reaction 1′.

These operating procedures eliminate triglycerides contained in high density lipoprotein and low density lipoprotein in a test sample and inhibit triglycerides contained in chylomicron in the test sample from being involved in the measurement reactions, whereby they derive hydrogen peroxide or a reduced coenzyme exclusively from triglycerides contained in very low density lipoprotein and intermediate density lipoprotein or in very low density lipoprotein and enable the selective measurement of such triglycerides.

I-2: First Step

The first step of the measurement method in accordance with this invention involves the following reactions:

1′. React lipoprotein lipase selectively with triglycerides contained in low density lipoprotein and high density lipoprotein in a test sample by exposing the test sample to lipoprotein lipase and some other enzymes, which catalyze a series of reactions leading to the generation of hydrogen peroxide or a reduced coenzyme from glycerol, in the presence of a first selective reaction promoter, which is an ether or ester compound of a polyoxyalkylene capable of reacting lipoprotein lipase selectively with triglycerides contained in low density lipoprotein and high density lipoprotein, to generate glycerol.

React the generated glycerol with enzymes that catalyze a series of reactions leading to the generation of hydrogen peroxide or a reduced coenzyme to derive hydrogen peroxide or a reduced coenzyme from the glycerol.

2′. React the hydrogen peroxide or reduced coenzyme generated by the above reaction 1′ with an enzyme which catalyzes a reaction leading to the conversion of hydrogen peroxide or a reduced coenzyme into another substance so that neither hydrogen peroxide nor reduced coenzyme is present in the reaction system.

3′. Convert the triglycerides contained in the low density lipoprotein and the high density lipoprotein into another substances by the above reactions 1′ and 2′ to eliminate the triglycerides from the reaction system.

I-3: Second Step

The second step of the measurement method in accordance with this invention involves the following reactions.

1′. Subsequently after the first step, expose the test sample to a second selective reaction promoter, which is capable of reacting lipoprotein lipase selectively with triglycerides contained in very low density lipoprotein, intermediate density lipoprotein, low density lipoprotein and high density lipoprotein, in the reaction system of the first step.

Then, react the test sample with lipoprotein lipase and some other enzymes, which catalyze a series of reactions leading to the generation of hydrogen peroxide or a reduced coenzyme from glycerol, in the presence of the second selective reaction promoter. Since the triglycerides contained in the low density lipoprotein and the high density lipoprotein have been already eliminated in the first step, only the triglycerides contained in the very low density lipoprotein and intermediate density lipoprotein or in the very low density lipoprotein react with lipoprotein lipase to generate glycerol.

React the generated glycerol with enzymes which catalyze a series of reaction leading to the generation of hydrogen peroxide or a reduced coenzyme from glycerol to derive hydrogen peroxide or a reduced coenzyme from the glycerol.

2′. Measure the hydrogen peroxide or reduced coenzyme generated by the above reaction 1′ directly or using some signal derived from the hydrogen peroxide or reduced coenzyme.

I-4. Selective Reaction Promoters

I-4-1: First Selective Reaction Promoter

The first selective reaction promoter used in this invention is a substance which is capable of reacting lipoprotein lipase selectively with triglycerides contained in low density lipoprotein and high density lipoprotein.

In other words, in the presence of the first selective reaction promoter, the reaction of lipoprotein lipase which leads to the generation of glycerol from triglycerides contained in lipoproteins occurs and progresses selectively in the triglycerides contained in low density lipoprotein and high density lipoprotein.

As the first selective reaction promoter, an ether compound or ester compound of a polyoxyalkylene is used.

Examples of polyoxyalkylene ether compounds suitably used as above include polyoxyalkylene straight-chain alkyl ethers (e.g. polyoxyethylene octyl ether, polyoxyethylene nonyl ether, polyoxyethylene lauryl ether, polyoxyethylene cetyl ether, polyoxyethylene stearyl ether, polyoxyethylene oleyl ether, polyoxyethylene behenyl ether), polyoxyalkylene branched-chain alkyl ethers, polyoxyalkylene straight-chain alkylphenyl ethers (e.g. polyoxyethylene octylphenyl ether, polyoxyethylene nonylphenyl ether, polyoxyethylene laurylphenyl ether, polyoxyethylene stearylphenyl ether), and polyoxyalkylene branched-chain alkylphenyl ethers.

Examples of polyoxyalkylene ester compounds suitably used as above include polyoxyalkylene straight-chain fatty acid esters (e.g. polyoxyalkylene laurate, polyoxyalkylene stearate, polyoxyalkylene oleate, polyoxyalkylene coconut oil fatty acid ester), polyoxyalkylene branched-chain fatty acid esters, polyoxyalkylene straight-chain alkyl substituted benzoic acid esters (e.g. polyoxyethylene octylbenzoate, polyoxyethylene nonylbenzoate, polyoxyethylene laurylbenzoate, polyoxyethylene stearylbenzoate,) and polyoxyalkylene branched-chain alkyl substituted benzoic acid esters.

Particularly suitably used are polyoxyalkylene straight-chain alkylphenyl ethers and polyoxyalkylene branched-chain alkylphenyl ethers.

The straight-chain alkyl groups and branched-chain alkyl groups in the above described polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain alkyl substituted benzoic acid esters and polyoxyalkylene branched-chain alkyl substituted benzoic acid esters as well as the straight-chain fatty acids and branched-chain fatty acids in the above described polyoxyalkylene straight-chain fatty acid esters and polyoxyalkylene branched-chain fatty acid esters are not limited to any specific ones; however, those of C₇₋₃₀ are preferably used.

The polyoxyalkylene group of the above described polyoxyalkylene ether or ester compounds is preferably a polyoxyethylene group.

Either one kind or two or more kinds of first selective reaction promoters may be selected from among the above described ones and used (present (contained)) in the reaction system.

The concentration of a first selective reaction promoter(s) present (contained) in the reaction system cannot be generalized, because it varies depending on the kind of the first selective reaction promoter(s) selected, the kind and origin of the enzymes which catalyze the series of reactions leading to the generation of hydrogen peroxide or a reduced coenzyme from triglycerides, the concentration of the triglycerides contained in lipoproteins in a test sample or, when measurement is made using a two-reagent system, the mixing ratio of the first reagent to the second reagent. Accordingly, the first selective reaction promoter may be present (contained) in the reaction system at a concentration suitable for such conditions. Generally a first selective reaction promoter(s) is present (contained) in the reaction system at a concentration of 0.005 to 5%, preferably 0.01 to 2% and particularly preferably 0.05 to 1%.

I-4-2: Second Selective Reaction Promoter

The second selective reaction promoter used in this invention is a substance which is capable of reacting lipoprotein lipase selectively with triglycerides contained in very low density lipoprotein, intermediate density lipoprotein, low density lipoprotein and high density lipoprotein.

In other words, in the presence of the second selective reaction promoter, the reaction of lipoprotein lipase which leads to the generation of glycerol from triglycerides contained in lipoproteins occurs and progresses selectively in the triglycerides contained in very low density lipoprotein, intermediate density lipoprotein, low density lipoprotein and high density lipoprotein.

As the second selective reaction promoter, an ether compound or ester compound of a polyoxyalkylene is suitably used.

Examples of polyoxyalkylene ether compounds suitably used as above include polyoxyalkylene straight-chain alkyl ethers (e.g. polyoxyethylene octyl ether, polyoxyethylene nonyl ether, polyoxyethylene lauryl ether, polyoxyethylene cetyl ether, polyoxyethylene stearyl ether, polyoxyethylene oleyl ether, polyoxyethylene behenyl ether), polyoxyalkylene branched-chain alkyl ethers, polyoxyalkylene straight-chain alkylphenyl ethers (e.g. polyoxyethylene octylphenyl ether, polyoxyethylene nonylphenyl ether, polyoxyethylene laurylphenyl ether, polyoxyethylene stearylphenyl ether), and polyoxyalkylene branched-chain alkylphenyl ethers.

Examples of polyoxyalkylene ester compounds suitably used as above include polyoxyalkylene straight-chain fatty acid esters (e.g. polyoxyalkylene laurate, polyoxyalkylene stearate, polyoxyalkylene oleate, polyoxyalkylene coconut oil fatty acid ester), polyoxyalkylene branched-chain fatty acid esters, polyoxyalkylene straight-chain alkyl substituted benzoic acid esters (e.g. polyoxyethylene octylbenzoate, polyoxyethylene nonylbenzoate, polyoxyethylene laurylbenzoate, polyoxyethylene stearylbenzoate,) and polyoxyalkylene branched-chain alkyl substituted benzoic acid esters.

Particularly suitably used are polyoxyalkylene straight-chain alkylphenyl ethers and polyoxyalkylene branched-chain alkylphenyl ethers.

The straight-chain alkyl groups and branched-chain alkyl groups in the above described polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain alkyl substituted benzoic acid esters and polyoxyalkylene branched-chain alkyl substituted benzoic acid esters as well as the straight-chain fatty acids and branched-chain fatty acids in the above described polyoxyalkylene straight-chain fatty acid esters and polyoxyalkylene branched-chain fatty acid esters are not limited to any specific ones; however, those of C₇₋₃₀ are preferably used.

The polyoxyalkylene group of the above described polyoxyalkylene ether or ester compounds is preferably a polyoxyethylene group.

Either one kind or two or more kinds of second selective reaction promoters may be selected from among the above described ones and used (present (contained)) in the reaction system.

The concentration of the second selective reaction promoter(s) present (contained) in the reaction system cannot be generalized, because it varies depending on the kind of the second selective reaction promoter(s) selected, the kind and source of the enzymes which catalyze the series of reactions leading to the generation of hydrogen peroxide or a reduced coenzyme from triglycerides, the concentration of the triglycerides contained in lipoproteins in a test sample or, when measurement is made using a two-reagent system, the mixing ratio of the first reagent to the second reagent. Accordingly, the second selective reaction promoter may be present (contained) in the reaction system at a concentration suitable for such conditions. The concentration of the second selective reaction promoter(s) present (contained) in the reaction system is usually 0.005 to 5%, preferably 0.01 to 2% and particularly preferably 0.05 to 1%.

In this invention, however, care should be taken not to allow the composition and concentration of the second selective reaction promoter in the second step and those of the first selective reaction promoter in the first step to be completely the same.

I-4-3: Average Mole Number of Added Polyoxyalkylene in Selective Reaction Promoters

Where the average mole number of the added polyoxyalkylene in a polyoxyalkylene ether compound or polyoxyalkylene ester compound as the first selective reaction promoter is m and that of the added polyoxyalkylene in a polyoxyalkylene ether compound or polyoxyalkylene ester compound as the second selective reaction promoter is n, it is preferable that the m/n ratio is in the range of 1.1 to 1.2.

It is more preferable that the m/n ratio is in the range of 1.1 to 1.2, m is in the range of 7.7 to 18 and n is in the range of 7 to 15. And it is particularly preferable that the m/n ratio is in the range of 1.1 to 1.2, m is in the range of 11 to 12 and n is 10.

It is also particularly preferable that the above described first selective reaction promoter is polyoxyethylene nonylphenyl ether in which the average mole number of the added polyoxyethylene m is in the range of 11 to 12 and the above described second selective reaction promoter is polyoxyethylene nonylphenyl ether in which the average mole number of the added polyoxyethylene n is 10.

The larger the value m becomes, the more selectively triglycerides contained in very low density lipoprotein can be measured than those contained in intermediate density lipoprotein.

The smaller the value m becomes, the more selectively triglycerides contained in intermediate density lipoprotein can be measured than those contained in very low density lipoprotein.

And the larger the m/n ratio becomes, the more selectively triglycerides contained in very low density lipoprotein can be measured than those contained in intermediate density lipoprotein.

Thus, the selectivity can be varied depending on the objective of the measurement by properly setting the value m or the m/n ratio.

I-5: Reactions Leading to the Generation of Hydrogen Peroxide or a Reduced Coenzyme from Triglycerides

I-5-1: Reactions Leading to the Generation of Hydrogen Peroxide or a Reduced Coenzyme from Triglycerides

In the measurement method of this invention, the reactions in the first and second steps which lead to the generation of hydrogen peroxide or a reduced coenzyme from triglycerides include: a reaction catalyzed by lipoprotein lipase; and a series of reactions leading to the generation of hydrogen peroxide and a reduced coenzyme from glycerol.

I-5-2: Reaction Catalyzed by Lipoprotein Lipase

The reaction catalyzed by lipoprotein lipase in this invention is a reaction leading to the hydrolysis of triglycerides contained in lipoproteins in a test sample into one molecule of glycerol and three molecules of fatty acid by exposing the triglycerides to lipoprotein lipase.

Any lipoprotein lipase can be used as long as it catalyzes the reaction leading to the hydrolysis of triglycerides into one molecule of glycerol and three molecules of fatty acid.

The lipoprotein lipase used in this invention may be derived from microorganisms such as bacteria or fungi, derived from animals such as human beings, pigs or cows, derived from plants, or prepared by the recombinant DNA technique.

The activity level of lipoprotein lipase present (contained) in the reaction system cannot be generalized, because it varies depending on the origin of the lipoprotein lipase, the kind of the first or second selective reaction promoter or, when measurement is made using a two-reagent system, the mixing ratio of the first reagent to the second reagent. Lipoprotein lipase may therefore be used at a level suitable for such conditions.

Generally preferably lipoprotein lipase is present (contained) in the reaction system at an activity level of 1 to 10,000,000 units/l and more preferably at an activity level of 100 to 1,000,000 units/l.

Normally, the activity level of an enzyme varies depending on the method employed for its measurement, and besides, even if the same method is employed for the same type of enzyme, the activity level obtained varies depending on the origin or the purity of the enzyme. Accordingly, even if the activity level of the lipoprotein lipase used is outside the above described range, the lipoprotein lipase can sometimes provide advantageous effects of this invention.

Preferably, the activity of the lipoprotein lipase present in the first step of the measurement method of this invention depends on the concentration of a surfactant, while that of the lipoprotein lipase present in the second step hardly depends on the concentration of a surfactant.

The “lipoprotein lipase whose activity depends on the concentration of a surfactant” means lipoprotein lipase whose activity increases with the increase in the concentration of a surfactant.

The “lipoprotein lipase whose activity hardly depends on the concentration of a surfactant” means lipoprotein lipase whose activity rapidly increases with the increase in the concentration of a surfactant and reaches a certain level, and from that point on, hardly changes even if the concentration of the surfactant increases.

Accordingly, discrimination between the above two types of lipoprotein lipase can be made by measuring the degree and pattern of increase in enzyme activity when increasing the concentration of a surfactant present in the reaction system.

One example of methods for this discrimination is shown in “Experimental example” described later.

For example, when dividing the measured value obtained in accordance with the method described in “Experimental example” by the measured value obtained when the reaction system contains lipoprotein lipase whose activity hardly depends on the concentration of a surfactant (e.g. LPL or LPL-311), if the quotient is 0.5 or less, the lipoprotein lipase is judged to be lipoprotein lipase whose activity depends on the concentration of a surfactant, whereas if the quotient is more than 0.5, the lipoprotein lipase is judged to be lipoprotein lipase whose activity hardly depends on the concentration of a surfactant.

Examples of the above described surfactants include nonionic surfactants, anionic surfactants, cationic surfactants and amphoteric surfactants. Particularly preferable are nonionic surfactants.

Examples of such nonionic surfactants include ether compounds or ester compounds of polyoxyalkylene. Particularly preferable are polyoxyalkylene branched-chain alkyl ether and polyoxyalkylene alkylphenyl formaldehyde condensates.

Examples of lipoprotein lipase whose activity depends on the concentration of a surfactant include “LP-BP” (Asahi Kasei Corp.) and “LPL-314” (Toyobo Co., Ltd.).

Examples of lipoprotein lipase whose activity hardly depends on the concentration of a surfactant include “LPL” (Asahi Kasei Corp.) and “LPL-311” (Toyobo Co., Ltd.).

I-5-3: A Series of Reactions Leading to the Generation of Hydrogen Peroxide or Reduced Coenzyme from Glycerol

The series of reactions leading to the generation of hydrogen peroxide or reduced coenzyme in this invention may be any reaction as long as it enables the generation of hydrogen peroxide or a reduced coenzyme from glycerol as a hydrolysis product by lipoprotein lipase. It may consist of a single reaction or a plurality of reactions.

Examples of reduced coenzymes include nicotinamide adenine dinucleotide (reduced form) [NADH (reduced)] and nicotinamide adenine dinucleotide phosphate (reduced form) [NADPH (reduced)].

One example of such series of reactions leading to the generation of hydrogen peroxide or a reduced coenzyme is a series of reactions that converts glycerol and adenosine triphosphate (ATP) into glycerol-3-phosphate and adenosine diphosphate (ADP) by the catalytic action of glycerol kinase and further converts the resultant glycerol-3-phosphate into dihydroxyacetone-3-phosphate, while generating hydrogen peroxide, by the catalytic action of glycerol-3-phosphate oxidase.

Another example of such series of reactions is a series of reactions that converts glycerol and adenosine triphosphate (ATP) into glycerol-3-phosphate and adenosine diphosphate (ADP) by the catalytic action of glycerol kinase and further converts the resultant glycerol-3-phosphate into dihydroxyacetone-3-phosphate, while generating nicotinamide adenine dinucleotide (reduced form) [NADH], in the presence of nicotinamide adenine dinucleotide (oxidized form) [NAD⁺] by the catalytic action of glycerol-3-phosphate dehydrogenase.

In the method of this invention, the enzymes that catalyze a series of reaction leading to the generation of hydrogen peroxide or a reduced coenzyme from glycerol may be any enzymes as long as they can catalyze the series of reaction leading to the generation of hydrogen peroxide or a reduced coenzyme from glycerol. Examples of such enzymes include: a combination of glycerol kinase and glycerol-3-phosphate oxidase, and a combination of glycerol kinase and glycerol-3-phosphate dehydrogenase.

The enzymes used in this invention may be derived from microorganisms such as bacteria or fungi, derived from animals such as human beings, pigs or cows, derived from plants, or prepared by the recombinant DNA technique.

The activity levels of the enzymes present (contained) in the reaction system cannot be generalized, because they vary depending on the kind and origin of the enzymes, the kind of the first or second selective reaction promoter or, when measurement is made using a two-reagent system, the mixing ratio of the first reagent to the second reagent. The enzymes may therefore be used at a level suitable for such conditions.

Generally preferably glycerol kinase is present (contained) in the reaction system at an activity level of 0.01 to 500,000 units/i and more preferably at an activity level of 10 to 10,000 units/l.

Also, generally preferably glycerol-3-phosphate oxidase is present (contained) in the reaction system at an activity level of 1 to 500,000 units/I and more preferably at an activity level of 100 to 50,000 units/l.

Normally, the activity level of an enzyme varies depending on the method employed for its measurement, and besides, even if the same method is employed for the same type of enzyme, the activity level obtained varies depending on the origin or the purity of the enzyme. Accordingly, even if the activity levels of the enzymes used are outside the above described range, the enzymes can sometimes provide advantageous effects of this invention.

The catalytic reaction by glycerol kinase requires adenosine triphosphate (ATP) and magnesium ion. Accordingly, when using glycerol kinase, adenosine triphosphate and magnesium ion are added to (or contained in) the reaction systems of the first and the second steps.

Adenosine triphosphate used may be in the form of a free acid or a salt. The concentration of adenosine triphosphate or the salt thereof present (contained) in the reaction system is generally preferably 0.001 to 50 g/l and particularly preferably 0.01 to 10 g/l.

The magnesium ion used in the reaction may be in the form of a salt with a halogen ion or an organic acid. It is generally preferably present (contained) in the reaction system at a concentration of 0.001 to 100 mM and particularly preferably at a concentration of 0.01 to 50 mM.

The catalytic reaction by glycerol-3-phosphate dehydrogenase requires an oxidized coenzyme such as nicotinamide adenine dinucleotide (oxidized form) [NAD⁺] or nicotinamide adenine dinucleotide phosphate (oxidized form) [NADP⁺ (oxidized)]. Accordingly, when using glycerol-3-phosphate dehydrogenase, the oxidized coenzyme is added to (or contained in) the reaction systems of the first and the second steps.

I-6: Reaction Leading to the Conversion of Hydrogen Peroxide or Reduced Coenzyme into another Substance

In the first step of the measurement method of this invention, hydrogen peroxide or a reduced coenzyme is generated from triglycerides contained in low density lipoprotein and high density lipoprotein in the presence of a first selective reaction promoter and the generated hydrogen peroxide or reduced coenzyme is converted into another substance.

This series of reactions leads to the elimination of triglycerides contained in low density lipoprotein and high density lipoprotein.

The reaction leading to the conversion of hydrogen peroxide or a reduced coenzyme into another substance may be any reaction as long as it can convert hydrogen peroxide or a reduced coenzyme into another substance. The reaction may consist of a single reaction or a plurality of reactions.

I-6-1: Reaction Leading to the Conversion of Hydrogen Peroxide into another Substance

I-6-1-1: Reaction by Catalase

One example of reactions leading to the conversion of hydrogen peroxide into another substance is a catalytic reaction by catalase that degrades two molecules of hydrogen peroxide to two molecules of water and one molecule of oxygen.

Any type of catalase may be used as long as it catalyzes the reaction leading to the degradation of two molecules of hydrogen peroxide to two molecules of water and one molecule of oxygen.

The catalase used in the reaction may be derived from microorganisms such as bacteria or fungi, derived from animals such as human beings, pigs or cows, derived from plants, or prepared by the recombinant DNA technique.

The activity level of catalase present (contained) in the reaction system cannot be generalized, because it varies depending on the origin of the catalase or, when measurement is made using a two-reagent system, the mixing ratio of the first reagent to the second reagent. Catalase may therefore be used at a level suitable for such conditions.

Generally preferably catalase is present (contained) in the reaction system at an activity level of 100 units/l or more.

Normally, the activity level of an enzyme varies depending on the method employed for its measurement, and besides, even if the same method is employed for the same type of enzyme, the activity level obtained varies depending on the origin or the purity of the enzyme. Accordingly, even if the activity level of the catalase used is outside the above described range, the catalase can sometimes provide advantageous effects of this invention.

After eliminating the generated hydrogen peroxide with catalase in the first step, it is necessary in the second step to deactivate the catalase so that hydrogen peroxide generated in the second step should not be eliminated (degraded) by the catalase.

This can be accomplished by addition of a substance that deactivate catalase, such as sodium azide, to the reaction system in the second step.

I-6-1-2: Reaction by Peroxidase

Another example of reactions leading to the conversion of hydrogen peroxide into another substance is a catalytic reaction by peroxidase (POD) that produces an oxidized substance from hydrogen peroxide and an oxidizable substance (e.g. a chromogen in a Trinder reaction system).

Any type of POD may be used as long as it catalyzes the reaction leading to the production of an oxidized substance from hydrogen peroxide and an oxidizable substance, and hence the consumption of hydrogen peroxide.

The POD used in the reaction may be derived from microorganisms such as bacteria or fungi, derived from animals such as human beings, pigs or cows, derived from plants such as horseradish, or prepared by the recombinant DNA technique.

The activity level of POD present (contained) in the reaction system cannot be generalized, because it varies depending on the origin of the POD or, when measurement is made using a two-reagent system, the mixing ratio of the first reagent to the second reagent. POD may therefore be added to (or contained in) the reaction system in the first step at a level suitable for such conditions.

Generally preferably POD is present (contained) in the reaction system at an activity level of 30 units/l or more.

Normally, the activity level of an enzyme varies depending on the method employed for its measurement, and besides, even if the same method is employed for the same type of enzyme, the activity level obtained varies depending on the origin or the purity of the enzyme. Accordingly, even if the activity level of the POD used is outside the above described range, the POD can sometimes provide advantageous effects of this invention.

Examples of substances to be oxidized include chromogens in a Trinder reaction system.

Examples of chromogens in a Trinder reaction system include 4-aminoantipyrine, phenol or derivatives thereof, or aniline derivatives.

If both 4-aminoantipyrine and phenol or derivative thereof or both 4-aminoantipyrine and an aniline derivative exist together in the reaction system of the first step, color is developed by the generated hydrogen peroxide and POD; therefore, preferably either 4-aminoantipyrine or phenol or derivative thereof alone, or either 4-aminoantipyrine or an aniline derivative alone is present (contained) in the reaction system.

The details of chromogens in a Trinder reaction system will be described later in I-7-1: Hydrogen peroxide (I-7: Measurement of hydrogen peroxide or reduced coenzyme).

I-6-2: Reaction Leading to the Conversion of Reduced Coenzyme into another Substance

One example of reactions leading to the conversion of a reduced coenzyme into another substance is a catalytic reaction by a dehydrogenase using the reduced coenzyme as a coenzyme that converts the reduced coenzyme into an oxidized coenzyme.

Any type of dehydrogenase may be used as long as it catalyzes the reaction leading to the conversion of the reduced coenzyme into an oxidized coenzyme using the reduced coenzyme as a coenzyme. Examples of such types of dehydrogenase include lactate dehydrogenase, malate dehydrogenase and isocitrate dehydrogenase.

The dehydrogenase used in the reaction may be derived from microorganisms such as bacteria or fungi, derived from animals such as human beings, pigs or cows, derived from plants, or prepared by the recombinant DNA technique.

The activity level of dehydrogenase present (contained) in the reaction system cannot be generalized, because it varies depending on the origin of the dehydrogenase or, when measurement is made using a two-reagent system, the mixing ratio of the first reagent to the second reagent. Dehydrogenase may therefore be added to the reaction system of the first step at a level suitable for such conditions.

I-7: Measurement of Hydrogen Peroxide or Reduced Coenzyme

In the second step of the measurement method of this invention, hydrogen peroxide or a reduced coenzyme is generated from triglycerides contained in very low density lipoprotein and intermediate density lipoprotein, or in very low density lipoprotein in the presence of a second selective reaction promoter and the generated hydrogen peroxide or reduced coenzyme is measured.

The measurement of triglycerides contained in very low density lipoprotein and intermediate density lipoprotein, or in very low density lipoprotein in a test sample is made through the series of reactions.

The method for measuring the hydrogen peroxide or reduced coenzyme generated in the second step may be any method as long as it enables the measurement of the amount or the presence of hydrogen peroxide or a reduced coenzyme generated through the above described series of reactions that leads to the generation of hydrogen peroxide or a reduced coenzyme from glycerol.

One example of such methods is a method in which some signal is derived from the generated hydrogen peroxide or reduced coenzyme.

I-7-1: Hydrogen Peroxide

To measure hydrogen peroxide, a hydrogen peroxide electrode or the like may be used to directly measure hydrogen peroxide or some signal may be derived from hydrogen peroxide to measure the signal.

One example of methods in which some signal is derived from hydrogen peroxide is a method utilizing a Trinder reaction system in which a chromogen is oxidized to generate a dye in the presence of POD and the absorbance of the generated dye is measured.

The POD used in the reaction may be derived from microorganisms such as bacteria or fungi, derived from animals such as human beings, pigs or cows, derived from plants such as horseradish, or prepared by the recombinant DNA technique.

Generally preferably the activity level of the POD present (or contained) in the reaction system is 30 units/l or more.

Examples of chromogens in a Trinder reaction system include the combination of 4-aminoantipyrine and phenol or derivative thereof, or the combination of 4-aminoantipyrine and an aniline derivative.

Generally preferably 4-aminoantipyrine is present (or contained) in the reaction system at a concentration of 0.001 to 50 g/l and particularly preferably at a concentration of 0.01 to 10 g/l.

Examples of phenol derivatives include 4-chlorophenol, 2,4-dichlorophenol, 2,4-dibromophenol, 2,4,6-trichlorophenol, and the salts thereof.

Examples of aniline derivatives include N-(2-hydroxy-3-sulfopropyl)-3,5-dimethoxyaniline (HDAOS), N-(3-sulfopropyl)-3,5-dimethoxyaniline (HDAPS), N-ethyl-N-(2-hydroxy-3-sulfopropyl)-3,5-dimethoxyaniline (DAOS), N-ethyl-N-(3-sulfopropyl)-3,5-dimethoxyaniline (DAOS), N-ethyl-N-(2-hydroxy-3-sulfopropyl)-3,5-dimethoxy-4-fluoroaniline (FDAOS), N-ethyl-N-(3-sulfopropyl)-3,5-dimethoxy-4-fluoroaniline (FDAPS), N-(2-carboxyethyl)-N-ethyl-3,5-dimethoxyaniline (CEDB), N-ethyl-N-(2-hydroxy-3-sulfopropyl)-3-methoxyaniline (ADOS), N-ethyl-N-(3-sulfopropyl)-3-methoxyaniline (ADPS), N-ethyl-N-(2-hydroxy-3-sulfopropyl)aniline (ALOS), N-ethyl-N-(3-sulfopropyl)aniline (ALPS), N-(3-sulfopropyl)aniline (HALPS), N-ethyl-N-(2-hydroxy-3-sulfopropyl)-3,5-dimethylaniline (MAOS), N-ethyl-N-(3-sulfopropyl)-3,5-dimethylaniline (MAPS), N-ethyl-N-(2-hydroxy-3-sulfopropyl)-3-methoxyaniline (TOOS), N-(2-carboxyethyl)-N-ethyl-3-methylaniline (CEMB), N-(2-carboxyethyl)-N-ethyl-3-methoxyaniline (CEMO) and the salts thereof.

Generally preferably the above described phenol or derivative thereof, or aniline derivative is present (or contained) in the reaction system at a concentration of 0.001 to 50 g/l and particularly preferably at a concentration of 0.01 to 10 g/l.

I-7-2: Reduced Coenzyme

Measurement of a reduced coenzyme may be made by, for example, measuring the absorbance of the reduced coenzyme itself, for example, at 340 nm or by deriving some other signal from the reduced coenzyme, followed by measuring the signal.

Examples of methods for deriving some signal from the reduced enzyme include a method utilizing a reaction that involves the reduction of a tetrazolium salt or the like in the presence of diaphorase, 1-methoxy-phenazine methosulfate or the like to produce a dye and measuring the dye.

I-8: Reaction Assistant

I-8-1: Reaction Assistant

In the measurement method of this invention, a reaction assistant may be present (or contained) in the reaction system of the first and/or the second step.

The presence (or addition) of a reaction assistant enhances the selective-reaction-promotion action of the first selective reaction promoter and/or the second selective reaction promoter.

Concrete examples of reaction assistants include polysaccharides or derivatives thereof, polyanions, halogen ions, metal ions, lectin and the like.

These reaction assistants may be present (or contained) in the reaction system in combination.

I-8-2: Polysaccharides or Derivatives Thereof

Examples of polysaccharides used in the measurement method of this invention include saccharides produced by dehydration condensation of several or more monosaccharides. Concrete examples are cyclodextrin (CD), dextran, heparin and the like.

Examples of the derivatives of polysaccharides include those produced by substituting hydrogen or a functional group such as hydroxyl group of the polysaccharides, with: hydroxypropyl group; hydroxybutyl group; glucosyl group; maltosyl group; diethylaminoethyl group; an aliphatic hydrocarbon group such as methyl or ethylidene; an alicyclic hydrocarbon group such as cyclopropyl; an aromatic hydrocarbon group such as phenyl, benzyl or benzylidene; an ether group such as methoxy or phenoxy; an ester group such as acetoxy or benzoyloxy; an acyl group such as acetyl, propionyl or benzoyl; a sulfhydryl group; a sulfo group; a sulfonyl group, a carboxyl group; an amino group; an imino group; a nitryl group; a carbonyl group; an oxo group; a hydroxyl group; or a nitro group.

Examples of the derivatives of polysaccharides also include crosslinked products of the above described polysaccharides or substituted polysaccharides.

Examples of the cyclodextrin used as the reaction assistant include α-cyclodextrin (α-CD), β-cyclodextrin (β-CD) and γ-cyclodextrin (γ-CD).

Examples of the cyclodextrin derivatives include: those produced by substituting a hydroxyl group of α-CD, β-CD or γ-CD with a group such as hydroxypropyl, hydroxybutyl, glucosyl, maltosyl, benzyl or sulfonyl; and crosslinked products of the above described cyclodextrin or derivatives thereof.

Of the above described types of cyclodextrin, β-CD or γ-CD is preferably used. Of the above described cyclodextrin derivatives, those produced by substituting a hydroxyl group of β-CD or γ-CD with a group as described above or crosslinked products of β-CD or γ-CD are preferably used.

When using dextran sulfate or derivative thereof, one whose molecular weight is in the range of 1,000 to 5,000,000 is preferably used and one whose molecular weight is in the range of 5,000 to 1,000,000 is particularly preferably used.

The concentration of polysaccharide or derivative thereof present (contained) in the reaction system cannot be generalized, because it varies depending on the kind of the polysaccharide or derivative selected, the kind and concentration of the first and/or second selective reaction promoter, the kind and origin of the enzymes which catalyze the series of reactions leading to the generation of hydrogen peroxide or a reduced coenzyme from triglycerides, the concentration of triglycerides contained in lipoproteins in a test sample or, when measurement is made using a two-reagent system, the mixing ratio of the first reagent to the second reagent. Accordingly, polysaccharide or derivative thereof may be added (or contained) in the reaction system at a concentration suitable for such conditions. Generally polysaccharide or derivative thereof may be present (contained) in the reaction system at a concentration of 0.005 to 5%, preferably at a concentration of 0.08 to 2% and particularly preferably 0.01 to 1%.

I-8-3: Other Reaction Assistants

Examples of polyanions used as the reaction assistants include phosphotungstate or the like.

Examples of halogen ions include a chloride ion or the like.

Examples of metal ions include divalent metal ions such as copper ion and manganese ion, or the like.

Examples of lectin used as the reaction assistants include lentil lectin or the like.

The concentration of a reaction assistant other than polysaccharide or derivative thereof present (contained) in the reaction system cannot be generalized, because it varies depending on the kind of the reaction assistant selected, the kind and concentration of the first and/or second selective reaction promoter, the kind and origin of the enzymes which catalyze the series of reactions leading to the generation of hydrogen peroxide or a reduced coenzyme from triglycerides, the concentration of triglycerides contained in lipoproteins in a test sample or, when measurement is made using a two-reagent system, the mixing ratio of the first reagent to the second reagent. Accordingly, a reaction assistant other than polysaccharide or derivative thereof may be added (or contained) in the reaction system at a concentration suitable for such conditions.

I-9: Other Substances

In the measurement method of this invention, additional substances may also be added to the reaction system, depending on the situation. Examples of additional substances include: buffers; enzymes other than the above described ones; substrates for the enzymes; coenzymes other than the above described ones; ions or salts of alkaline metals, alkaline earth metals or the like; chelators; proteins such as albumin; sodium azide, antibiotics or preservatives such as a synthetic antibacterial agent; stabilizers such as saccharides or polymer compounds; activators; substances involved in the elimination or inhibition of interfering substances contained in a test sample, such as ascorbate oxidase; excipients; and other reagent components.

Preferably the pH values of the reaction system in the first and second steps of the measurement method of this invention are in the range of 5 to 10 and more preferably in the range of 5.5 to 9.0.

Accordingly, it is preferable the reaction system in the first and second steps contains a buffer that allows the pH of the reaction system to be in the above described range.

Examples of such buffers include MES, Bis-Tris, Bis-Tris propane, ADA, PIPES, ACES, MOPSO, MOPS, BES, TES, HEPES, DIPSO, TAPSO, POPSO, HEPPSO, EPPS, Tricine, Bicine, TAPS, CHES, phosphoric acid, phosphate, boric acid, borate, glycine, glycylglycine, imidazole, and tris(hydroxymethyl)aminomethane[Tris].

I-10: Test Samples

In the measurement method of this invention, a test sample may be any test sample as long as it is suspected to include triglycerides contained in very low density lipoprotein and intermediate density lipoprotein, or in very low density protein and intended to be used for the measurement of triglycerides contained in very low density lipoprotein and intermediate density lipoprotein, or in very low density protein.

Examples of such test samples include: body fluids such as human or animal blood, serum and plasma; extracted fluids from human or animal organs or muscles; extracted fluids from human or animal feces; extracted fluids from cells and bacterial cells; and extracted fluids from plants.

I-11: Measurement Operations

The measurement method of this invention is to selectively measure triglycerides contained in very low density lipoprotein and intermediate density lipoprotein, or in very low density lipoprotein in a test sample in the above described first and second steps. However, the method may be carried out in two steps (two-step method, two-reagent system) or in three or more steps (multiple-step method, multiple-reagent system).

The measurement reaction may be initiated either by addition of a substrate or substances essential to the measurement reaction or by addition of a test sample.

The measurement operations may be performed in any set temperature range, such as at 30° C. or 37° C., where the reactions involved in the measurement progress and the ingredients involved in the reactions, such as enzymes, are not deactivated or degenerated by heat.

In the measurement method of this invention, the generated hydrogen peroxide or reduced coenzyme may be measured either by a reaction rate method or by an end-point method.

When the generated hydrogen peroxide or reduced coenzyme is measured by its absorbance or the like, a wavelength at which the measurement is made is appropriately selected from the ultraviolet, visible or infrared region depending on the type of substance to be measured.

The absorbance or the like may be measured at a single wavelength or at two wavelengths.

In the measurement method of this invention, measurement may be made either by hand or by using an instrument such as automatic analyzer.

Examples of such instruments include automatic analyzers for laboratory tests.

Examples of automatic analyzers for laboratory tests include: flow-type automatic analyzers such as continuous flow-type and flow injection-type analyzers; discrete-type automatic analyzers such as closed batch-type, open batch-type, pack-type and centrifuge-type analyzers; and dry chemistry-type automatic analyzers such as film-type and strip-type analyzers.

One example of measurement operations performed with an instrument will be shown below:

1′. introduce a measurement reagent used in the first step of the measurement method of this invention (a first reagent of this invention) and a measurement reagent used in the second step of the measurement method of this invention (a second reagent of this invention) into respective vessels adapted to the instrument used;

2′. place the vessels containing the respective reagents at given positions in the instrument;

3′. introduce a sample to be tested into a vessel adapted to the instrument and place it at a given position in the instrument;

4′. input and set measurement conditions (measurement parameters) for reagents used and a sample to be tested, when the instrument is an analyzer for laboratory tests;

5′. initiate the measurement;

generally, introduce the test sample and the first reagent each into a reaction cell (reaction cuvette) with a pipet (probe), tube or the like, mix, and expose them to each other to form a reaction system of the first step, and allow the reaction of the first step to progress while keeping the temperature constant,

6′. measure the absorbance at a specified wavelength for the reaction solution of the test sample and the first reagent (the reaction system of the first step) in the reaction cell (reaction cuvette), after a certain time has elapsed (after completion of the first step reactions);

7′. introduce the second reagent into the reaction solution in the reaction cell (reaction cuvette) with a pipet (probe), a tube or the like, mix and expose them to each other to form a reaction system of the second step, and allow the second step reaction to progress while keeping the temperature constant;

8′. measure the absorbance at a specified wavelength for the reaction solution of the test sample, the first reagent and the second reagent (the reaction system of the second step) in the reaction cell (reaction cuvette), after a certain time has elapsed (after completion of the second step reactions);

9′. perform the above operations 5′ to 8′ with a reaction system containing purified water instead of the above sample and measure the absorbance of the reagent blank;

10′. subtract the difference between the absorbance value obtained by the operation 6′ and the absorbance value of the reagent blank from the difference between the absorbance value obtained by the operation 8′ and the absorbance value of the reagent blank to obtain an absorbance difference; and

11′. compare the absorbance difference obtained by the operation 10′ with that of a triglyceride sample of known concentration (standard solution), calibration curve, and calculate the concentration of triglycerides in very low density lipoprotein and intermediate density lipoprotein, or in very low density lipoprotein in the test sample.

II. Reagents for Measurement

II-1: General Introduction of Reagents for Measurement

The reagent for selective measurement of triglycerides contained in very low density lipoprotein and intermediate density lipoprotein, or in very low density lipoprotein in a test sample in accordance with this invention is composed of a first reagent and a second reagent described below.

First Reagent:

A reagent that contains a first selective reaction promoter, which is an ether compound or ester compound of a polyoxyalkylene capable of reacting lipoprotein lipase selectively with triglycerides contained in low density lipoprotein and high density lipoprotein; lipoprotein lipase; enzymes which catalyze a series of reactions leading to the generation of hydrogen peroxide or a reduced coenzyme from glycerol; and an enzyme which catalyzes the reaction leading to the conversion of hydrogen peroxide or a reduced coenzyme into another substance, and if necessary, further contains a substance involved in the reaction leading to the derivation of some signal from hydrogen peroxide or a reduced coenzyme.

Second Reagent:

A reagent that contains a second selective reaction promoter, which is capable of reacting lipoprotein lipase selectively with triglycerides contained in very low density lipoprotein, intermediate density lipoprotein, low density lipoprotein and high density lipoprotein, and if necessary, further contains a substance involved in the reaction leading to the derivation of some signal from hydrogen peroxide or a reduced coenzyme.

The reagent for measurement in accordance with this invention is composed of the above described first and second reagents; however, it may be combined with other reagents and/or a standard substance (calibrated substance).

In the reagent in accordance with this invention, the ingredients of the first reagent may be divided into two or more groups and used as two or more different reagents (in this case, the first reagent is composed of two or more reagents).

Similarly, the ingredients of the second reagent may be divided into two or more groups and used as two or more different reagents (in this case, the second reagent is composed of two or more reagents).

The reagent having such a composition makes it possible to eliminate triglycerides contained in high density lipoprotein and low density lipoprotein in a test sample and inhibit triglycerides contained in chylomicron in the same sample from being involved in the measurement reactions at the time of measuring the test sample, whereby triglycerides contained in very low density lipoprotein and intermediate density lipoprotein or in very low density lipoprotein alone are degraded to hydrogen peroxide or a reduced coenzyme and can be selectively measured.

II-2: First Reagent

The first reagent constituting the measurement reagent in accordance with this invention is used for performing the first step of the measurement method of this invention (the details of the first step have already been described in “I-2: First step”).

II-3: Second Reagent

The second reagent constituting the measurement reagent in accordance with this invention is used for performing the second step of the measurement method of this invention (the details of the second step have already been described in “I-3: Second step”).

II-4: Selective Reaction Promoter

II-4-1: First Selective Reaction Promoter

The first selective reaction promoter which is contained in the first reagent constituting the measurement reagent in accordance with this invention is as described in “I-4-1: First selective reaction promoter”.

II-4-2: Second Selective Reaction Promoter

The second selective reaction promoter which is contained in the second reagent constituting the measurement reagent in accordance with this invention is as described in “I-4-2: Second selective reaction promoter”.

II-4-3: Average Mole Number of Added Polyoxyalkylene in Selective Reaction Promoters

The average mole number of the added polyoxyalkylene in the first selective reaction promoter contained in the first reagent which constitutes the measurement reagent in accordance with this invention and in the second selective reaction promoter contained in the second reagent which constitutes the measurement reagent in accordance with this invention are as described in “I-4-3: Average mole number of added polyoxyalkylene in selective reaction promoters”

II-5: Lipoprotein Lipase

The lipoprotein lipase which is contained in the first reagent constituting the measurement reagent of this invention is as described in “I-5-2: Reaction catalyzed by lipoprotein lipase”.

Lipoprotein lipase may be contained not only in the first reagent but also in both of the first reagent and the second reagent.

II-6: Enzyme Catalyzing a Series of Reactions Leading to the Generation of Hydrogen Peroxide or a Reduced Coenzyme from Glycerol

The enzyme which is contained in the first reagent constituting the measurement reagent of this invention and catalyzes a series of reactions leading to the generation of hydrogen peroxide or a reduced coenzyme from glycerol may be any enzyme as long as it catalyzes the reactions leading to the generation of hydrogen peroxide or a reduced coenzyme from glycerol generated by the hydrolysis by lipoprotein lipase. The enzyme may be composed of a single kind of enzyme that catalyzes the series of reactions or composed of more than one kind of enzyme that is involved in the series of reactions.

Examples of enzymes catalyzing the series of reactions leading to the generation of hydrogen peroxide or a reduced coenzyme from glycerol include glycerol kinase, glycerol-3-phosphate oxidase and glycerol-3-phosphate dehydrogenase.

The details of the series of reactions leading to the generation of hydrogen peroxide or a reduced coenzyme from glycerol and those of the enzymes catalyzing the reactions are as described in “I-5-3: A series of reactions leading to the generation of hydrogen peroxide or reduced coenzyme from glycerol”.

The enzyme catalyzing a series of reactions leading to the generation of hydrogen peroxide or a reduced coenzyme from glycerol may be contained not only in the first reagent but also in both of the first reagent and the second reagent.

The catalytic reaction by glycerol kinase requires adenosine triphosphate (ATP) and magnesium ion. Accordingly, when using glycerol kinase, adenosine triphosphate and magnesium ion are present (or contained) in the first reagent.

The details of adenosine triphosphate (ATP) and magnesium ion are as described in “I-5-3: A series of reactions leading to the generation of hydrogen peroxide or reduced coenzyme from glycerol”

The adenosine triphosphate (ATP) or the magnesium ion may be contained not only in the first reagent but also in both of the first reagent and the second reagent.

The catalytic reaction by glycerol-3-phosphate dehydrogenase requires an oxidized coenzyme such as nicotinamide adenine dinucleotide (oxidized form) [NAD⁺] or nicotinamide adenine dinucleotide phosphate (oxidized form) [NADP⁺ (oxidized)]. Accordingly, when using glycerol-3-phosphate dehydrogenase, the oxidized coenzyme is added to the first reagent (or the first and second reagents).

II-7: Enzymes Catalyzing the Reaction Leading to the Conversion of Hydrogen Peroxide or a Reduced Coenzyme into another Substance

The enzyme which is contained in the first reagent constituting the measurement reagent of this invention and catalyzes a reaction leading to the conversion of hydrogen peroxide or a reduced coenzyme into another substance may be any enzyme as long as it can catalyze the reaction leading to the conversion of the generated hydrogen peroxide or a reduced coenzyme into another substance. The enzyme may be composed of a single kind of enzyme that catalyzes the reaction or composed of more than one kind of enzyme that is involved in the reaction.

Examples of enzymes which catalyze the reaction leading to the conversion of hydrogen peroxide into another substance include catalase and peroxidase.

Examples of enzymes which catalyze the reaction leading to the conversion of a reduced coenzyme into another substance include dehydrogenase, which catalyzes the reaction leading to the conversion of the reduced coenzyme into an oxidized coenzyme using the reduced coenzyme as a coenzyme. More specifically, they include lactate dehydrogenase, malate dehydrogenase and isocitrate dehydrogenase.

When adding catalase to the first reagent and using it as an enzyme that catalyzes the reaction leading to the conversion of hydrogen peroxide into another substance, a substance that inhibits the activity of catalase, such as sodium azide, should be added to the second reagent so that the hydrogen peroxide generated in the second step is not eliminated (degraded) by the catalase.

When adding peroxidase to the first reagent and using it as an enzyme that catalyzes the reaction leading to the conversion of hydrogen peroxide into another substance, an oxidizable substance also needs be added to the first reagent.

This allows hydrogen peroxide and the oxidizable substance to be converted into oxidized substances through the catalytic reaction by peroxidase.

Examples of oxidizable substances include 4-aminoantipyrine, phenol or derivatives thereof, or aniline derivatives.

If both 4-aminoantipyrine and phenol or derivative thereof or both 4-aminoantipyrine and an aniline derivative exist together in the first reagent, color is developed in the first step by the generated hydrogen peroxide and peroxidase; therefore, either 4-aminoantipyrine or phenol or derivative thereof alone, or either 4-aminoantipyrine or an aniline derivative alone must be present (contained) in the first reagent.

The details of the reaction leading to the conversion of hydrogen peroxide or a reduced coenzyme into another substance and those of the enzyme catalyzing the reaction are as described in “I-6: Reaction leading to the conversion of hydrogen peroxide or reduced coenzyme into another substance”.

II-8: Substances Involved in the Reaction Leading to the Derivation of some Signal from Hydrogen Peroxide or Reduced Coenzyme

Substances involved in the reaction leading to the derivation of some signal from hydrogen peroxide or reduced coenzyme may be added, depending on the situation, to the first reagent, the second reagent, or both the first and second reagents which constitute the measurement reagent of this invention.

In measurement of the hydrogen peroxide or reduced coenzyme which is generated by mixing and catalytically reacting a test sample with the first reagent (the first step) and further mixing and catalytically reacting the test sample with the second reagent (the second step), except in case the generated hydrogen peroxide itself or the generated reduced coenzyme itself is measured, a substance involved in the reaction leading to the derivation of some signal from the hydrogen peroxide or reduced coenzyme needs to be contained in the first reagent, the second reagent, or both the first and second reagents.

The substance involved in the reaction leading to the derivation of some signal from the hydrogen peroxide or reduced coenzyme may be any substance as long as it is involved in the reaction leading to the derivation of some signal from the generated hydrogen peroxide or reduced coenzyme. The substance may be composed of a single substance involved in the above described reaction or composed of more than one substance involved in the above described reaction.

Examples of substances involved in the reaction leading to the derivation of some signal from hydrogen peroxide or a reduced coenzyme include peroxidase, chromogens in a Trinder reaction system (4-aminoantipyrine, phenol or derivatives thereof, and aniline derivatives), diaphorase, 1-methoxy-phenazine methosulfate, and tetrazolium salts.

All the substances involved in the reaction leading to the derivation of some signal from hydrogen peroxide or a reduced coenzyme may be added to one reagent, either the first reagent or the second reagent; however, from the viewpoint of the substance stability, preferable they are distributed between the two reagents.

For example, the following two substances: 4-aminoantipyrine and phenol or derivative thereof, or 4-aminoantipyrine and an aniline derivative are separated from each other and distributed between the first and second reagents.

If the following three substances: peroxidase, 4-aminoantipyrine and phenol or derivative thereof, or peroxidase, 4-aminoantipyrine and an aniline derivative are contained together in the first reagent, color is developed by the generated hydrogen peroxide in the first step; therefore, preferably the above described three substances are distributed between the first and second reagents.

The details of the substances involved in the reaction leading to the derivation of some signal from hydrogen peroxide or a reduced coenzyme is as described in “I-7: Measurement of hydrogen peroxide or reduced coenzyme”.

II-9: Reaction Assistant

In the measurement reagent of this invention, the first and/or the second reagent may contain a reaction assistant.

The details of the reaction assistant are as described in “I-8: Reaction assistant”.

II-10: Other substances

The first and/or the second reagent constituting the measurement reagent of this invention may contain, depending on the situation, additional substances such as buffers; enzymes other than the above described ones; substrates for the other enzymes; coenzymes other than the above described ones; ions or salts of alkaline metals, alkaline earth metals or the like; chelators; proteins such as albumin; sodium azide, antibiotics or preservatives such as a synthetic antibacterial agent; stabilizers such as saccharides or polymer compounds; activators; substances involved in the elimination or inhibition of measurement interfering substances contained in a test sample, such as ascorbate oxidase; excipients; and other reagent ingredients.

Preferably the pH values of the first and the second reagents constituting the measurement reagent of this invention are in the range of 5 to 10 and particularly preferably in the range of 5.5 to 9.0.

Accordingly, it is preferable the first and the second reagents each contain a buffer that allows their pH values to fall in the above described range.

Examples of such buffers are as described in “I-9: Other substances”.

II-11: Test Sample

The details of the samples to be tested with the measurement reagent of this invention are as described in “I-10: Test samples”.

II-12: Measurement operations

The details of the operations for selective measurement, using the measurement reagent of this invention, of triglycerides contained in very low density lipoprotein and intermediate density lipoprotein or in very low density lipoprotein in the test sample are as described in “I-11: Measurement operations”.

This specification includes part or all of the contents as disclosed in the specification of Japanese Patent Application No. 2002-168738, which is a priority document of the present application.

### BEST MODE FOR CARRYING OUT THE INVENTION

The present invention will be described in more detail giving several examples. The examples are not intended to limit the scope of this invention.

### EXAMPLE 1

Measurement of triglycerides in purified lipoprotein fractions using the measurement method and reagent of this invention

Triglycerides in purified lipoprotein fractions were measured using the measurement method and reagent of this invention while varying substances used as the first selective reaction promoter and the second selective reaction promoter.

1. Preparation of Measurement Reagent of this Invention

1-1: Preparation of First Reagent (Reagent A)

The following reagent ingredients were dissolved in pure water to give the respective indicated concentrations, thereby preparing Reagent A of pH 6.0 (20° C.).

| Reagent ingredient | Concentation | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |
| Lipoprotein lipase [LP-BP (Asahi Kasei Corporation)] | 300,000 | units/l |
| First selective reaction promoter [name of each | 0.1% | (w/v) |
| substance is shown in Table 1] | | |

TABLE 1

| | Supplier | Chemical structure |
|---|---|---|
| First selective | | |
| reaction promoter | | |
| NP-10 | Nikko Chemicals Co., Ltd. | Polyoxyethylene nonylphenyl ether (10) |
| Emulgen 911 | Kao Corporation | Polyoxyethylene nonylphenyl ether (11) |
| NP-11.2 | Nikko Chemicals Co., Ltd. | Polyoxyethylene nonylphenyl ether (11.2) |
| NP-11.4 | Nikko Chemicals Co., Ltd. | Polyoxyethylene nonylphenyl ether (11.4) |
| NP-11.5 | Nikko Chemicals Co., Ltd. | Polyoxyethylene nonylphenyl ether (11.5) |
| NP-11.6 | Nikko Chemicals Co., Ltd. | Polyoxyethylene nonylphenyl ether (11.6) |
| NP-13 | Nikko Chemicals Co., Ltd. | Polyoxyethylene nonylphenyl ether (13) |
| Second selective | | |
| reaction promoter | | |
| NP-10 | Nikko Chemicals Co., Ltd. | Polyoxyethylene nonylphenyl ether (10) |
| NP-11.2 | Nikko Chemicals Co., Ltd. | Polyoxyethylene nonylphenyl ether (11.2) |
| NP-11.4 | Nikko Chemicals Co., Ltd. | Polyoxyethylene nonylphenyl ether (11.4) |
| NP-11.6 | Nikko Chemicals Co., Ltd. | Polyoxyethylene nonylphenyl ether (11.6) |
| NP-13 | Nikko Chemicals Co., Ltd. | Polyoxyethylene nonylphenyl ether (13) |

*The values in parentheses indicate the average mole number of the added polyoxyethylene.

1-2: Preparation of Second Reagent (Reagent B)

The following reagent ingredients were dissolved in pure water to give the respective indicated concentrations, thereby preparing Reagent B of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL (Asahi Kasei Corporation)] | 500,000 | units/l |
| Sodium azide | 0.1% | (w/v) |
| Second selective reaction promoter [name of each | 0.5% | (w/v) |
| substance is shown in Table 1] | | |

2. Preparation of Reagent for Total Triglyceride Measurement (Control)

2-1: Preparation of First Reagent (Reagent C)

The following reagent ingredients were dissolved in pure water to give the respective indicated concentrations, thereby preparing Reagent C of pH 6.0 (20° C.).

| Reagent ingredient | Concentration | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |
| Adekanol B-795 (Asahi Denka Kogyo K.K.) | 0.5% | (w/v) |

2-2: Preparation of Second Reagent (Reagent D)

The following reagent ingredients were dissolved in pure water to give the respective indicated concentrations, thereby preparing Reagent D of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL (Asahi Kasei Corporation)] | 120,000 | units/l |
| Sodium azide | 0.1% | (w/v) |
| Adekanol B-795 (Asahi Denka Kogyo K.K.) | 0.5% | (w/v) |

3. Preparation of Purified Lipoprotein Fractions

Blood was collected in a tube containing an anticoagulant and separated by density-gradient centrifugation into 5 types of lipoprotein fractions having different densities: chylomicron, very low density lipoprotein, intermediate density lipoprotein, low density lipoprotein and high density lipoprotein. These five types of fractions were used as test samples in the measurement of triglycerides.

4. Measurement of Triglycerides in Lipoprotein Fractions

The level of triglycerides in each lipoprotein fraction prepared in section 3 was measured using a Hitachi automatic analyzer 7170S (Hitachi, Ltd.) through the following procedure:

1′. Dispense 2.5 μl of each lipoprotein fraction prepared in section 3, as a test sample, in a reaction cell (reaction cuvette).

Then, dispense 200 μl of the first reagent of this invention (Reagent A) prepared in section 1-1 in the reaction cell (reaction cuvette) and mix it with the test sample.

Warm up the reaction cell (reaction cuvette) at 37° C. for the reaction of the first step.

2′. Four minutes 30 seconds (16th point) after the addition of the first reagent (Reagent A), measure the absorbance (main wavelength 600 nm, sub wavelength 700 nm) of the reaction solution (the reaction solution of the test sample and the first reagent) in the reaction cell (reaction cuvette) to give a sample blank.

3′. After the measurement of the absorbance, dispense 100 μl of the second reagent of this invention (Reagent B) prepared in section 1-2 in the reaction solution in the reaction cell (reaction cuvette) and mix it with the reaction solution in the cell (cuvette).

Then, warm up the reaction cell (reaction cuvette) at 37° C. for the reaction of the second step.

4′. Nine minutes 47 seconds (34th point) after the addition of the first reagent (Reagent A), measure the absorbance (main wavelength 600 nm, sub wavelength 700 nm) of the reaction solution (the reaction solution of the test sample, the first reagent and the second reagent) in the reaction cell (reaction cuvette) to give a measured value of the test sample.

5′. Repeat the operating procedures 1′ to 4′ for the reaction solution in which the test sample has been replaced by purified water to give a measured absorbance of a reagent blank.

6′. Calculate the absorbance difference of the test sample by subtracting the difference between the absorbance obtained in procedure 2′ (sample blank) and the absorbance of the reagent blank from the difference between the absorbance obtained in procedure 4′ (measured value) and the absorbance of the reagent blank.

7′. Perform the same operating procedures as 1′ to 6′ using: an aqueous solution of triolein (trioleoylglycerol) (concentration: 250 mg/dl), which is a kind of triglyceride, as a test sample; the first reagent of the total triglyceride measurement reagent (Reagent C) prepared in 2-1; and the second reagent of the total triglyceride measurement reagent (Reagent D) prepared in 2-2 to calculate the absorbance difference of the aqueous solution of triolein [calibration curve].

8′. Compare the absorbance difference of the test sample calculated in the operating procedure 6′ with that of the aqueous solution of triolein (250 mg/dl) calculated in procedure 7′ [calibration curve] and calculate the concentration of triglycerides contained in the test sample (the lipoprotein fraction).

The combinations of the first selective reaction promoter contained in the first reagent (Reagent A) and the second selective reaction promoter contained in the second reagent (Reagent B) used in the measurement of triglycerides are shown in Table 2.

The level of triglycerides contained in each lipoprotein fraction prepared in 3 was measured using the first reagent of the total triglyceride measurement reagent prepared in 2-1 (Reagent C) and the second reagent of the total triglyceride measurement reagent prepared in 2-2 (Reagent D) in the same manner as described above.

The effect of the first and the second selective reaction promoters added into the measurement reagent of this invention was confirmed by the values obtained by dividing the level (measured value) of triglycerides contained in each test sample (lipoprotein fraction) when using the measurement reagent of this invention (Reagent A and Reagent B) by the level (measured value) of triglycerides contained in each test sample (lipoprotein fraction) when using the total triglyceride measurement reagent (Reagent C and Reagent D).

The values are shown in Table 2.

TABLE 2

| | Second | | | | | |
|---|---|---|---|---|---|---|
| First selective | selective | | | | | |
| reaction | reaction | | | | | |
| promoter | promoter | | | | | |
| contained in first | contained in | | | | | |
| reagent | second reagent | Test sample (Lipoprotein fraction) | | | | |
| (Reagent A) | (Reagent B) | CM | VLDL | IDL | LDL | HDL |
| NP-10 | NP-10 | 0.05 | 0.01 | 0.03 | 0.04 | 0.01 |
| Emulgen 911 | NP-10 | 0.08 | 0.69 | 0.63 | 0.13 | 0.03 |
| NP-11.2 | NP-10 | 0.07 | 0.48 | 0.44 | 0.07 | 0.01 |
| NP-11.4 | NP-10 | 0.07 | 0.62 | 0.56 | 0.09 | 0.03 |
| NP-11.5 | NP-10 | 0.08 | 0.69 | 0.64 | 0.18 | 0.03 |
| NP-11.6 | NP-10 | 0.08 | 0.69 | 0.64 | 0.17 | 0.02 |
| NP-13 | NP-10 | 0.08 | 0.72 | 0.65 | 0.50 | 0.04 |
| Emulgen 911 | NP-10 | 0.07 | 0.61 | 0.49 | 0.07 | 0.01 |
| Emulgen 911 | NP-11.2 | 0.03 | 0.29 | 0.35 | 0.06 | 0.00 |
| Emulgen 911 | NP-11.4 | 0.03 | 0.26 | 0.32 | 0.05 | 0.02 |
| Emulgen 911 | NP-11.6 | 0.03 | 0.23 | 0.28 | 0.05 | 0.02 |
| Emulgen 911 | NP-13 | 0.03 | 0.16 | 0.17 | 0.04 | 0.02 |

| | | | | | |
|---|---|---|---|---|---|
| Reagents for measuring total | 1.00 | 1.00 | 1.00 | 1.00 | 1.00 |
| triglyceride (Reagent C and | | | | | |
| Reagent D) [Control] | | | | | |

CM: Chylomicron
VLDL: Very low density lipoprotein
IDL: Intermediate density lipoprotein
LDL: Low density lipoprotein
HDL: High density lipoprotein

### 5. SUMMARY

5-1: When the Second Selective Reaction Promoter is NP-10

The measured results will be described below which were obtained using NP-10 (polyoxyethylene nonylphenyl ether in which the average mole number of the added polyoxyethylene is 10) as the second selective reaction promoter contained in the second reagent (Reagent B) of this invention while varying the average mole number of the added polyoxyethylene in the first selective reaction promoter (polyoxyethylene nonylphenyl ether) contained in the first reagent of this invention (Reagent A).

1′. The results show that when the average mole number of the added polyoxyethylene in the first selective reaction promoter was 10 [NP-10], triglycerides contained in each lipoprotein fraction could not be measured in any one of the lipoprotein fractions: chylomicron fraction, very low density lipoprotein fraction, intermediate density lipoprotein fraction, low density lipoprotein fraction and high density lipoprotein fraction.

2′. The results also show that when the average mole number of the added polyoxyethylene in the first selective reaction promoter was 11 [Emulgen 911], 11.2 [NP-11.2], 11.4 [NP-11.4], 11.5 [NP-11.5] or 11.6 [NP-11.6], triglycerides could be measured in the lipoprotein fractions such as very low density lipoprotein fraction and intermediate density lipoprotein fraction, while they was hardly measured or only a very small amount of them was measured in the lipoprotein fractions such as chylomicron fraction, low density lipoprotein fraction and high density lipoprotein fraction.

This confirms that where the average mole number of the added polyoxyethylene is rounded to the nearest whole number, when the average mole number (n) of the added polyoxyethylene in the second selective reaction promoter is 10 and the average mole number (m) of the added polyoxyethylene in the first selective reaction promoter is 11 to 12 (when the m/n ratio is 1.1 to 1.2), triglycerides contained in very low density lipoprotein and intermediate density lipoprotein can be selectively measured, compared with those contained in chylomicron, low density lipoprotein and high density lipoprotein.

3′. The results show that when the average mole number of the added polyoxyethylene in the first selective reaction promoter was 13 [NP-13], not only triglycerides contained in the very low density lipoprotein fraction and the intermediate density lipoprotein fraction, but triglycerides contained in the low density lipoprotein fraction were measured.

This confirms that with NP-13, triglycerides contained in very low density lipoprotein and intermediate density lipoprotein or in very low density lipoprotein cannot be selectively measured.

5-2: When the First Selective Reaction Promoter is Emulgen 911

The measured results will be described below which were obtained using Emulgen 911 (polyoxyethylene nonylphenyl ether in which the average mole number of the added polyoxyethylene is 11) as the first selective reaction promoter contained in the first reagent (Reagent A) of this invention while varying the average mole number of the added polyoxyethylene in the second selective reaction promoter (polyoxyethylene nonylphenyl ether) contained in the second reagent of this invention (Reagent B).

1′. The results show that when the average mole number of the added polyoxyethylene in the second selective reaction promoter was 10 [NP-10], triglycerides contained in the very low density lipoprotein fraction and the intermediate density lipoprotein fraction could be measured, while triglycerides contained in the chylomicron fraction, the low density lipoprotein fraction and the high density lipoprotein fraction could hardly be measured.

The experimental results also confirms that when the average mole number (m) of the added polyoxyethylene in the first selective reaction promoter is 11 and the average mole number (n) of the added polyoxyethylene in the second selective reaction promoter is 10 (when the m/n ratio is 1.1), triglycerides contained in very low density lipoprotein and intermediate density lipoprotein can be selectively measured, compared with those contained in chylomicron, low density lipoprotein and high density lipoprotein.

2′. The results also show that when the average mole number of the added polyoxyethylene in the second selective reaction promoter was 11.2 [NP-11.2], 11.4 [NP-11.4], 11.6 [NP-11.6] or 13 [NP-13], triglycerides could be measured in the lipoprotein fractions such as very low density lipoprotein fraction and intermediate density lipoprotein fraction; however, the percentage was low.

This confirms that where the average mole number of the added polyoxyethylene is rounded to the nearest whole number, the measurement reagents in which the average mole number (m) of the added polyoxyethylene in the first selective reaction promoter is 11 and the average mole number (n) of the added polyoxyethylene in the second selective reaction promoter is 11 to 13 (when the m/n ratio is 0.85 to 1.0) are not suitably used for the selective measurement of triglycerides contained in very low density lipoprotein and intermediate density lipoprotein or in very low density lipoprotein.

### EXAMPLE 2

Confirmation of Effect of Reaction Assistants

The effect of the presence (addition) of a reaction assistant on the selective-reaction-promotion activity of the first selective reaction promoter and/or the second selective reaction promoter was confirmed.

1. Preparation of Measurement Reagent of this Invention

1-1: Preparation of First Reagent (Reagent E)

The following reagent ingredients were dissolved in pure water to give the respective indicated concentrations, thereby preparing Reagent E of pH 6.0 (20° C.).

| Reagent ingredient | Concentration | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |
| Lipoprotein lipase [LP-BP (Asahi Kasei Corporation)] | 100,000 | units/l |
| Emulgen 911 (Kao Corporation) | 0.1% | (w/v) |

Reaction assistants (the substance name and the concentration of each assistant is shown in Table 3)

TABLE 3

| Reaction assistant | Chemical structure | Concentration |
|---|---|---|
| β-CD | β-cyclodextrin | 0.01% |
| | | 0.05% |
| | | 0.10% |
| γ-CD | γ-cyclodextrin | 0.01% |
| | | 0.05% |
| | | 0.10% |
| Hydroxypropyl-γ-CD | Hydroxypropyl-γ-cyclodextrin | 0.01% |
| (Nihon Shokuhin Kako Co., Ltd.) | | 0.05% |
| | | 0.10% |
| Sodium α-CD sulfonate | Sodium α-cyclodextrin sulfonate | 0.01% |
| | | 0.05% |
| | | 0.10% |
| ISOELEAT P | The total amount of cyclodextrin | 0.01% |
| (Bio Research Corporation of | constitutes 80% or more of ISOELEAT P | 0.05% |
| Yokohama) | and 50% or more of the total cyclodextrin | 0.10% |
| | is maltosyl cyclodextrin. | |
| Maltosyl-β-CD | Maltosyl-β-cyclodextrin | 0.01% |
| | | 0.05% |
| | | 0.10% |
| Water-soluble β-cyclodextrin | Water-soluble β-cyclodextrin polymer | 0.01% |
| polymer | (epichlorohydrin crosslinked) | 0.05% |
| (Nihon Shokuhin Kako Co., Ltd.) | | 0.10% |
| Water-soluble γ-cyclodextrin | Water-soluble γ-cyclodextrin polymer | 0.01% |
| polymer | (epichlorohydrin crosslinked) | 0.05% |
| (Nihon Shokuhin Kako Co., Ltd.) | | 0.10% |

None (no reaction assistant added)

1-2: Preparation of Second Reagent (Reagent F)

The following reagent ingredients were dissolved in pure water to give the respective indicated concentrations, thereby preparing Reagent F of pH 6.0 (20° C.).

| Reagent ingredient | Concentration | |
|---|---|---|
| 2-Morpholinoethanesulfonic acid [MES] | 50 | mM |
| 4-aminoantipyrine | 0.75 | mM |
| Peroxidase | 600 | units/l |
| Lipoprotein lipase [LPL (Asahi Kasei Corporation)] | 500,000 | units/l |
| Sodium azide | 0.1% | (w/v) |
| NP-10 (Nikko Chemicals Co., Ltd.) | 0.5% | (w/v) |

2. Preparation of Reagent for Total Triglyceride Measurement (Control)

2-1: Preparation of First Reagent (Reagent C)

The first reagent (Reagent C) prepared in Example 1, section 2-1 was used.

2-2: Preparation of Second Reagent (Reagent D)

The second reagent (Reagent D) prepared in Example 1, section 2-2 was used.

3. Purified lipoprotein fractions

The five types of lipoprotein fractions: chylomicron, very low density lipoprotein, intermediate density lipoprotein, low density lipoprotein and high density lipoprotein fractions prepared in Example 1, section 3 were used as test samples in the measurement of triglycerides.

4. Measurement of Triglycerides in Lipoprotein Fractions

The level of triglycerides in each lipoprotein fraction prepared in section 3 was measured through the same operating procedures as procedures 1′ to 8′ described in Example 1, section 4, except that the first reagent of this invention (Reagent A) was replaced by the first reagent of this invention (Reagent E) prepared in 1-1 and the second reagent of this invention (Reagent B) was replaced by the second reagent of this invention (Reagent F) prepared in 1-2.

Measurement of triglycerides was made for the cases using the above described kinds of first reagent (Reagent E) (for the cases using the above described reaction assistants and concentrations).

The level of triglycerides contained in the lipoprotein fractions prepared in section 3 was also measured in the same manner as above, using the first reagent of the total triglyceride measurement reagent (Reagent C) prepared in section 2-1 and the second reagent of the total triglyceride measurement reagent (Reagent D) prepared in section 2-2.

The level (measured value) of triglycerides in each test sample (lipoprotein fraction) measured using the measurement reagent of this invention (Reagent E and Reagent F) was divided by the level (measured value) of triglycerides in the same test sample (lipoprotein fraction) measured using the total triglyceride measurement reagent (Reagent C and Reagent D).

The values are shown in Table 4.

TABLE 4

| | Concen- | Test sample (Lipoprotein fraction) | | | | |
|---|---|---|---|---|---|---|
| Reaction assistant | tration | CM | VLDL | IDL | LDL | HDL |
| β-CD | 0.01% | 0.05 | 0.72 | 0.61 | 0.23 | 0.03 |
| | 0.05% | 0.05 | 0.67 | 0.54 | 0.15 | −0.04 |
| | 0.1% | 0.05 | 0.59 | 0.49 | 0.18 | 0.02 |
| γ-CD | 0.01% | 0.05 | 0.72 | 0.60 | 0.33 | 0.01 |
| | 0.05% | 0.05 | 0.70 | 0.56 | 0.19 | 0.02 |
| | 0.1% | 0.05 | 0.63 | 0.52 | 0.18 | 0.03 |
| Hydroxypropyl-γ-CD | 0.01% | 0.05 | 0.72 | 0.62 | 0.22 | 0.04 |
| (Nihon Shokuhin Kako | 0.05% | 0.05 | 0.71 | 0.58 | 0.20 | 0.04 |
| Co., Ltd.) | 0.1% | 0.05 | 0.67 | 0.53 | 0.16 | 0.03 |
| Sodium α-CD sulfonate | 0.01% | 0.05 | 0.73 | 0.61 | 0.21 | 0.01 |
| | 0.05% | 0.05 | 0.72 | 0.62 | 0.21 | 0.04 |
| | 0.1% | 0.05 | 0.72 | 0.61 | 0.21 | 0.02 |
| ISOELEAT P | 0.01% | 0.05 | 0.72 | 0.61 | 0.25 | 0.04 |
| (Bio Research | 0.05% | 0.05 | 0.72 | 0.59 | 0.23 | 0.05 |
| Corporation of | 0.1% | 0.05 | 0.71 | 0.59 | 0.19 | 0.06 |
| Yokohama) | | | | | | |
| Maltosyl-β-CD | 0.01% | 0.05 | 0.72 | 0.61 | 0.25 | 0.04 |
| | 0.05% | 0.05 | 0.69 | 0.57 | 0.19 | 0.04 |
| | 0.1% | 0.05 | 0.58 | 0.50 | 0.18 | 0.05 |
| Water-soluble | 0.01% | 0.05 | 0.74 | 0.63 | 0.25 | 0.06 |
| β-cyclodextrin polymer | 0.05% | 0.05 | 0.69 | 0.57 | 0.18 | 0.04 |
| (Nihon Shokuhin Kako | 0.1% | 0.05 | 0.60 | 0.52 | 0.17 | 0.04 |
| Co., Ltd.) | | | | | | |
| Water-soluble | 0.01% | 0.05 | 0.75 | 0.61 | 0.26 | 0.05 |
| γ-cyclodextrin polymer | 0.05% | 0.05 | 0.72 | 0.60 | 0.22 | 0.05 |
| (Nihon Shokuhin Kako | 0.1% | 0.05 | 0.69 | 0.57 | 0.19 | 0.03 |
| Co., Ltd.) | | | | | | |

| | | | | | |
|---|---|---|---|---|---|
| None (no reaction assistant added) | 0.05 | 0.72 | 0.61 | 0.28 | 0.04 |
| Reagents for measuring total | 1.00 | 1.00 | 1.00 | 1.00 | 1.00 |
| triglyceride (Reagent C and | | | | | |
| Reagent D) [Control] | | | | | |

CM: Chylomicron
VLDL: Very low density lipoprotein
IDL: Intermediate density lipoprotein
LDL: Low density lipoprotein
HDL: High density lipoprotein

### 5. SUMMARY

1′. The results indicate that the addition of β-CD as a reactant assistant into the first reagent of this invention (Reagent E) made possible the decrease in degree to which triglycerides contained in low density lipoprotein were measured.

The results also indicate that although the increase in the concentration of β-CD decreased the degree to which triglycerides contained in very low density lipoprotein and intermediate density lipoprotein were measured, the rate of the decrease was less than that in the case of triglycerides in low density lipoprotein measured.

The use of hydroxypropyl-γ-CD, maltosyl-β-CD, water-soluble β-cyclodextrin polymer or water-soluble γ-cyclodextrin polymer as a reaction assistant produced the same effects.

2′. The results indicate that the addition of γ-CD as a reaction assistant into the first reagent of this invention (Reagent E) made possible the decrease in degree to which triglycerides contained in low density lipoprotein were measured.

Although the increase in the concentration of γ-CD decreased the degree to which triglycerides contained in very low density lipoprotein and intermediate density lipoprotein were measured, the rate of the decrease was less than that in the case of triglycerides in low density lipoprotein measured.

3′. The results indicate that the addition of sodium α-CD sulfonate as a reaction assistant into the first reagent of this invention (Reagent E) made possible the decrease in degree to which triglycerides contained in low density lipoprotein were measured.

4′. The results also indicate that the addition of ISOELEAT P as a reaction assistant into the first reagent of this invention (Reagent E) made possible the decrease in degree to which triglycerides contained in low density lipoprotein were measured.

The increase in the concentration of ISOELEAT P added also made possible the decrease in degree to which triglycerides contained in low density lipoprotein were measured.

The results described so far confirm that the presence (addition) of cyclodextrin or derivative thereof as a reaction assistant makes it possible to enhance the selective-reaction-promotion activity of the first selective reaction promoter and/or the second selective reaction promoter.

### EXAMPLE 3

Confirmation of Effects of Lipoprotein Lipase's Character

The effects of characters of of the lipoprotein lipase contained in the first reagent (present in the first step) and the lipoprotein lipase contained in the second reagent (present in the second step) on the measurement results were confirmed.

1. Preparation of Measurement Reagent of this Invention

1-1: Preparation of First Reagent (Reagent G)

The following reagent ingredients were dissolved in pure water to give the respective indicated concentrations, thereby preparing Reagent G of pH 6.0 (20° C.).

| Reagent ingredient | Concentration | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |
| Emulgen 911 (Kao Corporation) | 0.1% | (w/v) |

Lipoprotein lipase [the trade name and the activity level of each lipase are shown in Table 5]

1-2: Preparation of Second Reagent (Reagent H)

The following reagent ingredients were dissolved in pure water to give the respective indicated concentrations, thereby preparing Reagent H of pH 6.0 (20° C.).

| Reagent ingredient | Concentration | |
|---|---|---|
| 2-Morpholinoethanesulfonic acid [MES] | 50 | mM |
| 4-aminoantipyrine | 0.75 | mM |
| Peroxidase | 600 | units/l |
| Sodium azide | 0.1% | (w/v) |
| NP-10 (Nikko Chemicals Co., Ltd.) | 0.5% | (w/v) |

Lipoprotein lipase [the trade name and the activity level of each lipase are shown in Table 5]

2. Preparation of Reagent for Total Triglyceride Measurement (Control)

2-1: First Reagent (Reagent C)

The first reagent (Reagent C) prepared in Example 1, section 2-1 was used.

2-2: Second Reagent (Reagent D)

The second reagent (Reagent D) prepared in Example 1, section 2-2 was used.

3. Purified Lipoprotein Fractions

The five types of lipoprotein fractions: chylomicron, very low density lipoprotein, intermediate density lipoprotein, low density lipoprotein and high density lipoprotein prepared in Example 1, section 3 were used as test samples in the measurement of triglycerides.

4. Measurement of Triglycerides in Lipoprotein Fractions

The level of triglycerides in each lipoprotein fraction prepared in section 3 was measured through the same operating procedures as procedures 1′ to 8′ described in Example 1, section 4, except that the first reagent of this invention (Reagent A) was replaced by the first reagent of this invention (Reagent G) prepared in section 1-1 and the second reagent of this invention (Reagent B) was replaced by the second reagent of this invention (Reagent H) prepared in section 1-2.

The combinations of the lipoprotein lipase contained in the first reagent of this invention (Reagent G) and the lipoprotein lipase contained in the second reagent of this invention (Reagent H) in the measurement as above are as shown in Table 5.

The level of triglycerides in each lipoprotein fraction prepared in section 3 was also measured in the same manner as above, using the first reagent of the total triglyceride measurement reagent (Reagent C) in section 2-1 and the second reagent of the total triglyceride measurement reagent (Reagent D) in section 2-2.

The level (measured value) of triglycerides in each test sample (lipoprotein fraction) measured using the measurement reagent of this invention (Reagent G and Reagent H) was divided by the level (measured value) of triglycerides in the same test sample (lipoprotein fraction) measured using the total triglyceride measurement reagent (Reagent C and Reagent D).

The values are shown in Table 5.

TABLE 5

| Lipoprotein lipase | Lipoprotein lipase | | | | | |
|---|---|---|---|---|---|---|
| contained in the first | contained in the second | Test sample (lipoprotein fraction) | | | | |
| reagent (Reagent G) | reagent (Reagent H) | CM | VLDL | IDL | LDL | HDL |
| LP-BP [50,000 units/l] | LPL [500,000 units/l] | 0.02 | 0.67 | 0.55 | 0.30 | 0.03 |
| LP-BP [100,000 units/l] | LPL [500,000 units/l] | 0.02 | 0.66 | 0.55 | 0.22 | 0.03 |
| LP-BP [250,000 units/l] | LPL [500,000 units/l] | 0.02 | 0.68 | 0.56 | 0.18 | 0.03 |
| LP-BP [500,000 units/l] | LPL [500,000 units/l] | 0.01 | 0.66 | 0.53 | 0.15 | 0.01 |
| LPL-314 [100 units/l] | LPL [500,000 units/l] | 0.03 | 0.64 | 0.55 | 0.28 | 0.05 |
| LPL-314 [500 units/l] | LPL [500,000 units/l] | 0.02 | 0.63 | 0.49 | 0.17 | 0.02 |
| LPL-314 [1,000 units/l] | LPL [500,000 units/l] | 0.02 | 0.63 | 0.45 | 0.14 | 0.03 |
| LP-BP [50,000 units/l] | LPL-314 [100 units/l] | 0.01 | 0.46 | 0.47 | 0.28 | 0.03 |
| LP-BP [50,000 units/l] | LPL-314 [500 units/l] | 0.01 | 0.49 | 0.48 | 0.27 | 0.01 |
| LP-BP [50,000 units/l] | LPL-314 [1,000 units/l] | 0.02 | 0.50 | 0.49 | 0.28 | 0.03 |
| LPL-311 [100 units/l] | LPL [500,000 units/l] | 0.04 | 0.64 | 0.55 | 0.28 | 0.06 |
| LPL-311 [500 units/l] | LPL [500,000 units/l] | 0.02 | 0.59 | 0.41 | 0.15 | 0.01 |
| LPL-311 [1,000 units/l] | LPL [500,000 units/l] | 0.02 | 0.48 | 0.30 | 0.12 | 0.03 |

| | | | | | |
|---|---|---|---|---|---|
| Total triglyceride measurementreagents(Reagent | 1.00 | 1.00 | 1.00 | 1.00 | 1.00 |
| C and Reagent D)[control] | | | | | |

* Lipoprotein lipase whose activity depends on the presence of a surfactant.
LP-BP (Asahi Kasei Corporation)
LPL-314 (Toyobo Co., Ltd.)
* Lipoprotein lipase whose activity hardly depends on the presence of a surfactant.
LPL (Asahi Kasei Corporation)
LPL-311 (Toyobo Co., Ltd.)
CM: Chylomicron
VLDL: Very low density lipoprotein
IDL: Intermediate density lipoprotein
LDL: Low density lipoprotein
HDL: High density lipoprotein

### 5. SUMMARY

1′. The results indicate that when LP-BP (Asahi Kasei Corporation), a lipoprotein lipase whose activity depends on the concentration of a surfactant, was contained in the first reagent (present in the first step) and LPL (Asahi Kasei Corporation), a lipoprotein lipase whose activity hardly depends on the concentration of a surfactant, was contained in the second reagent (present in the second step), triglycerides contained in the very low density lipoprotein fractions and intermediate density lipoprotein fractions could be measured, while triglycerides contained in the chylomicron fractions, low density lipoprotein fractions and high density lipoprotein fractions could hardly be measured or be measured only in a very small amount.

The results also indicate that the degree to which triglycerides contained in low density lipoprotein fractions were measured was decreased with the increase in the activity level of LP-BP contained in the first reagent (present in the first step).

2′. The results indicate that when LPL-314 (Toyobo Co., Ltd.), a lipoprotein lipase whose activity depends on the concentration of a surfactant, was contained in the first reagent (present in the first step) and LPL (Asahi Kasei Corporation), a lipoprotein lipase whose activity hardly depends on the concentration of a surfactant, was contained in the second reagent (present in the second step), triglycerides contained in the very low density lipoprotein fractions and intermediate density lipoprotein fractions could be measured, while triglycerides contained in the chylomicron fractions, low density lipoprotein fractions and high density lipoprotein fractions could hardly be measured or be measured only in a very small amount.

The results also indicate that the degree to which triglycerides contained in low density lipoprotein fractions were measured was decreased with the increase in the activity level of LPL-314 contained in the first reagent (present in the first step).

3′. The results indicate that when LP-BP (Asahi Kasei Corporation), a lipoprotein lipase whose activity depends on the concentration of a surfactant, was contained in the first reagent (present in the first step) and LPL-314 (Toyobo Co., Ltd.), also a lipoprotein lipase whose activity depends on the concentration of a surfactant, was contained in the second reagent (present in the second step), triglycerides contained in the very low density lipoprotein fractions and intermediate density lipoprotein fractions could be measured, while triglycerides contained in the chylomicron fractions, low density lipoprotein fractions and high density lipoprotein fractions could be measured only in a very small amount.

However, the results show that the degree to which triglycerides contained in very low density lipoprotein fractions and intermediate density lipoprotein fractions were measured was a little decreased, compared with that of 1′ and 2′.

4′. The results indicate that when LPL-311 (Toyobo Co., Ltd.), a lipoprotein lipase whose activity hardly depends on the concentration of a surfactant, was contained in the first reagent (present in the first step) and LPL (Asahi Kasei Corporation), also a lipoprotein lipase whose activity hardly depends on the concentration of a surfactant, was contained in the second reagent (present in the second step), triglycerides contained in the very low density lipoprotein fractions and intermediate density lipoprotein fractions could be measured, while triglycerides contained in the chylomicron fractions, low density lipoprotein fractions and high density lipoprotein fractions could hardly be measured or be measured only in a very small amount.

However, the result also indicate that the degree to which triglycerides contained in the very low density lipoprotein fractions and intermediate density lipoprotein fractions were measured was decreased with the increase in the activity level of LPL-311 contained in the first reagent (present in the first step).

The results described so far confirm that lipoprotein lipase contained in the first reagent (present in the first step) is preferably of a type whose activity depends on the concentration of a surfactant and lipoprotein lipase contained in the second reagent (present in the second step) is preferably of a type whose activity hardly depends on the concentration of a surfactant.

### EXPERIMENTAL EXAMPLE

Confirmation of Lipoprotein Lipase's Character That the activity depends on the concentration of a surfactant or that the activity hardly depends on the concentration of a surfactant was confirmed for different types of lipoprotein lipase.

1. Preparation of Measurement Reagent

(Preparation of First Reagents)

1-1: Preparation of First Reagent (Reagent I)

The following reagent ingredients were dissolved in pure water to give the respective indicated concentrations, thereby preparing Reagent I of pH 6.0 (20° C.).

| Reagent ingredient | Concentration | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |

| | |
|---|---|
| BT-12 [polyoxyethylene secondary alkyl ether in | 0.1% (w/v) |
| which the average mole number of the added | |
| polyoxyethylene is 12] (Nikko Chemicals Co., Ltd) | |

1-2: Preparation of First Reagent (Reagent J)

The following reagent ingredients were dissolved in pure water to give the respective indicated concentrations, thereby preparing Reagent J of pH 6.0 (20° C.).

| Regent ingredient | Concentration | |
|---|---|---|
| 2-Morpholinoethanesulfonic acid [MES] | 50 | mM |
| N-(2-hydroxy-3-sulfopropyl)-3,5-dimethoxyaniline | 1.5 | mM |
| sodium salt (chromogen) | | |
| Glycerol kinase | 150 | units/l |
| Glycerol-3-phosphate oxidase | 3,000 | units/l |
| Sodium adenosine triphosphate | 0.5 | mM |
| Magnesium chloride hexahydrate | 1 | mM |
| Catalase | 100,000 | units/l |

| | |
|---|---|
| R-1020 [polyoxyethylene nonylphenyl formaldehyde | 0.1% (w/v) |
| condensate] (Nikko Chemicals Co., Ltd) | |

(Preparation of Second Reagents)

1-3: Preparation of Second Reagent (Reagent K)

The following reagent ingredients were dissolved in pure water to give the respective indicated concentrations, thereby preparing Reagent K of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LP-BP (Asahi Kasei Corporation)] | 100,000 | units/l |

| | |
|---|---|
| BT-12 [polyoxyethylene secondary alkyl ether in | 0.1% (w/v) |
| which the average mole number of the added | |
| polyoxyethylene is 12] (Nikko Chemicals Co., Ltd) | |

1-4: Preparation of Second Reagent (Reagent L)

The following reagent ingredients were dissolved in pure water to give the respective indicated concentrations, thereby preparing Reagent L of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL-314 (Toyobo Co., Ltd.)] | 100 | units/l |

| | |
|---|---|
| BT-12 [polyoxyethylene secondary alkyl ether in | 0.1% (w/v) |
| which the average mole number of the added | |
| polyoxyethylene is 12] (Nikko Chemicals Co., Ltd) | |

1-5: Preparation of Second Reagent (Reagent M)

The following reagent ingredients were dissolved in pure water to give the respective indicated concentrations, thereby preparing Reagent M of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL (Asahi Kasei corporation)] | 100,000 | units/l |

| | |
|---|---|
| BT-12 [polyoxyethylene secondary alkyl ether in | 0.1% (w/v) |
| which the average mole number of the added | |
| polyoxyethylene is 12] (Nikko Chemicals Co., Ltd) | |

1-6: Preparation of Second Reagent (Reagent N)

The following reagent ingredients were dissolved in pure water to give the respective indicated concentrations, thereby preparing Reagent N of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL-311 (Toyobo Co., Ltd.)] | 100 | units/l |

| | |
|---|---|
| BT-12 [polyoxyethylene secondary alkyl ether in | 0.1% (w/v) |
| which the average mole number of the added | |
| polyoxyethylene is 12] (Nikko Chemicals Co., Ltd) | |

1-7: Preparation of Second Reagent (Reagent 0)

The following reagent ingredients were dissolved in pure water to give the respective indicated concentrations, thereby preparing Reagent 0 of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LP-BP (Asahi Kasei Corporation)] | 100,000 | units/l |

| | |
|---|---|
| R-1020 [polyoxyethylene nonylphenyl formaldehyde | 0.1% (w/v) |
| condensate] (Nikko Chemicals Co., Ltd) | |

1-8: Preparation of Second Reagent (Reagent P)

The following reagent ingredients were dissolved in pure water to give the respective indicated concentrations, thereby preparing Reagent P of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL-314 (Toyobo Co., Ltd.)] | 100 | units/l |

| | |
|---|---|
| R-1020 [polyoxyethylene nonylphenyl formaldehyde | 0.1% (w/v) |
| condensate] (Nikko Chemicals Co., Ltd) | |

1-9: Preparation of Second Reagent (Reagent Q)

The following reagent ingredients were dissolved in pure water to give the respective indicated concentrations, thereby preparing Reagent Q of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL (Asahi Kasei Corporation)] | 100,000 | units/l |

| | |
|---|---|
| R-1020 [polyoxyethylene nonylphenyl formaldehyde | 0.1% (w/v) |
| condensate] (Nikko Chemicals Co., Ltd) | |

1-10: Preparation of Second Reagent (Reagent R)

The following reagent ingredients were dissolved in pure water to give the respective indicated concentrations, thereby preparing Reagent R of pH 6.0 (20° C.).

| Reagent ingredient | Concentration | |
|---|---|---|
| 2-Morpholinoethanesulfonic acid [MES] | 50 | mM |
| 4-Aminoantipyrine | 0.75 | mM |
| Peroxidase | 600 | units/l |
| Lipoprotein lipase [LPL-311 (Toyobo Co., Ltd)] | 100 | units/l |

| | |
|---|---|
| R-1020 [polyoxyethylene nonylphenyl formaldehyde | 0.1% (w/v) |
| condensate] (Nikko Chemicals Co., Ltd) | |

2. Preparation of Purified Lipoprotein Fractions

Blood was collected in a tube containing an anticoagulant and subjected to density-gradient centrifugation to obtain a very low density lipoprotein fraction. This fraction was used as a test sample in the measurement of triglycerides.

3. Confirmation of Lipoprotein Lipase's Character

The character of each lipoprotein lipase was confirmed by measuring, using the reagents prepared in section 1, triglycerides contained in the very low density lipoprotein fraction prepared in section 2.

The measurement was made for the cases using the respective reagent combinations shown in Table 6 in the same manner as in Example 1, section 4 “Measurement of triglycerides in lipoprotein fractions” using a Hitachi automatic analyzer 7170S (Hitachi, Ltd.).

To confirm the character of each lipoprotein lipase, each of the measured values [level of triglycerides contained in each test sample (very low density lipoprotein fraction)] “measured value” was divided by another measured value as shown in Table 6.

The values are shown in Table 6.

TABLE 6
Combinations of reagents

| | First | Second reagent |
|---|---|---|
| | reagent | (Lipoprotein lipase) |
| Measurement 1 | Reagent I | Reagent K (LP-BP) |
| Measurement 2 | Reagent I | Reagent L (LPL-314) |
| Measurement 3 | Reagent I | Reagent M (LPL) |
| Measurement 4 | Reagent I | Reagent N (LPL-311) |
| Measuratuion 5 | Reagent J | Reagent O (LP-BP) |
| Measurement 6 | Reagent J | Reagent P (LPL-314) |
| Measurement 7 | Reagent J | Reagent Q (LPL) |
| Measurement 8 | Reagent J | Reagent R (LPL-311) |

Values obtained by dividing a measured
value by another measured value
Surfactant: BT-12

| | |
|---|---|
| Measurement 1/measurement | 0.15 |
| 3(LP-BP/LPL) | |
| Measurement 2/measurement | 0.44 |
| 4(LP-314/LP-311) | |

Surfactant: R-1020

| | |
|---|---|
| Measurement 5/measurement | 0.46 |
| 7(LP-BP/LPL) | |
| Measurement 6/measurement | 0.29 |
| 8(LP-314/LP-311) | |

### 4. SUMMARY

4-1: When Surfactant is BT-12

1′. When dividing the measured value obtained using a measurement reagent whose second reagent contained LP-BP by the measured value obtained using a measurement reagent whose second reagent contained LPL, the quotient was 0.15.

This indicates that the enzymatic activity of LPL was higher than that of LP-BP when both measurement reagents contained the surfactant at the same concentration.

This confirms that LP-BP is lipoprotein lipase of a type whose activity depends on the concentration of the surfactant, while LPL is lipoprotein lipase of a type whose activity hardly depends on the concentration of the surfactant.

2′. When dividing the measured value obtained using a measurement reagent whose second reagent contained LPL-314 by the measured value obtained using a measurement reagent whose second reagent contained LPL-311, the quotient was 0.44.

This indicates that the enzymatic activity of LPL-311 was higher than that of LPL-314 when both measurement reagents contained the surfactant at the same concentration.

This confirms that LPL-314 is lipoprotein lipase of a type whose activity depends on the concentration of the surfactant, while LPL-311 is lipoprotein lipase of a type whose activity hardly depends on the concentration of the surfactant.

4-2: When Surfactant is R-1020

1′. When dividing the measured value obtained using a measurement reagent whose second reagent contained LP-BP by the measured value obtained using a measurement reagent whose second reagent contained LPL, the quotient was 0.46.

This indicates that the enzymatic activity of LPL was higher than that of LP-BP when both measurement reagents contained the surfactant at the same concentration.

This confirms that LP-BP is lipoprotein lipase of a type whose activity depends on the concentration of the surfactant while LPL is lipoprotein lipase of a type whose activity hardly depends on the concentration of the surfactant.

2′. When dividing the measured value obtained using a measurement reagent whose second reagent contained LPL-314 by the measured value obtained using a measurement reagent whose second reagent contained LPL-311, the quotient was 0.29.

This indicates that the enzymatic activity of LPL-311 was higher than that of LPL-314 when both measurement reagents contained the surfactant at the same concentration.

This confirms that LPL-314 is lipoprotein lipase of a type whose activity depends on the concentration of the surfactant, while LPL-311 is lipoprotein lipase of a type whose activity hardly depends on the concentration of the surfactant.

All publications, patents and patent applications cited herein are incorporated herein by reference in their entirety.

### INDUSTRIAL APPLICABILITY

The method and reagent for selective measurement of triglycerides contained in very low density lipoprotein and intermediate density lipoprotein or in very low density lipoprotein in a test sample in accordance with this invention do not require labor and time consuming operation, such as separating operation by ultracentrifuge, and is applicable to automatic analyzers widely in use, thereby providing more readily and accurately performable measurement.

1. A method for selective measurement of triglycerides contained in very low density lipoprotein and intermediate density lipoprotein or in very low density lipoprotein in a test sample, comprising the following two steps:
a first step which comprises
1′. exposing and reacting the test sample to and with lipoprotein lipase and some other enzymes, which catalyze a series of reactions leading to the generation of hydrogen peroxide or a reduced coenzyme from glycerol, in the presence of a first selective reaction promoter, which is an ether or ester compound of a polyoxyalkylene capable of reacting lipoprotein lipase selectively with triglycerides contained in low density lipoprotein and high density lipoprotein, to generate hydrogen peroxide or a reduced coenzyme from the triglycerides contained in the low density lipoprotein and the high density lipoprotein in the test sample,
2′. reacting the hydrogen peroxide or reduced coenzyme generated by the reaction 1′ with an enzyme which catalyzes a reaction leading to the conversion of hydrogen peroxide or a reduced coenzyme into another substance, and
3′. eliminating the triglycerides contained in the low density lipoprotein and the high density lipoprotein by the reactions of 1′ and 2′,
and
a second step which comprises
1′. subsequently, after the first step, reacting the test sample with lipoprotein lipase and some other enzymes, which catalyze a series of reactions leading to the generation of hydrogen peroxide or a reduced coenzyme from glycerol, in the presence of a second selective reaction promoter, which is capable of reacting lipoprotein lipase selectively with triglycerides contained in very low density lipoprotein, intermediate density lipoprotein, low density lipoprotein and high density lipoprotein, to generate hydrogen peroxide or a reduced coenzyme from the triglycerides contained in the very low density lipoprotein and the intermediate density lipoprotein or in the very low density lipoprotein, and
2′. measuring the hydrogen peroxide or reduced coenzyme generated by the reaction 1′.

2. The method according to claim 1, wherein the second selective reaction promoter is an ether or ester compound of a polyoxyalkylene.

3. The method according to claim 2, wherein m/n ratio is in the range of 1.1 to 1.2, where m is the average mole number of the added polyoxyalkylene in its ether or ester compound which is used as the first selective reaction promoter and n is the average mole number of the added polyoxyalkylene in its ether or ester compound which is used as the second selective reaction promoter.

4. The method according to claim 3, wherein m is in the range of 7.7 to 18 and n is in the range of 7 to 15.

5. The method according to claim 3, wherein m is in the range of 11 to 12 and n is 10.

6. The method according to claim 1, wherein the ether or ester compound of a polyoxyalkylene which is used as the first selective reaction promoter is at least one selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

7. The method according to claim 1, wherein the second selective reaction promoter is at least one ether or ester compound of a polyoxyalkylene selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

8. The method according to claim 1, wherein the polyoxyalkylene is polyoxyethylene.

9. The method according to claim 1, wherein the first selective reaction promoter is polyoxyethylene nonylphenyl ether in which the average mole number of added polyoxyethylene m is in the range of 11 to 12 and the second selective reaction promoter is polyoxyethylene nonylphenyl ether in which the average mole number of added polyoxyethylene n is 10.

10. The method according to claim 1, wherein the first step and/or the second step is carried out in the presence of a reaction assistant.

11. The method according to claim 10, wherein the reaction assistant is a polysaccharide or derivative thereof, a polyanion, a halogen ion, a metal ion, or lectin.

12. The method according to claim 1, wherein the activity of the lipoprotein lipase being present in the first step depends on the concentration of a surfactant, while that of the lipoprotein lipase being present in the second step hardly depends on the concentration of a surfactant.

13. A reagent for selective measurement of triglycerides contained in very low density lipoprotein and intermediate density lipoprotein or in very low density lipoprotein in a test sample, comprising
a first reagent that comprises: a first selective reaction promoter, which is an ether or ester compound of a polyoxyalkylene capable of reacting lipoprotein lipase selectively with triglycerides contained in low density lipoprotein and high density lipoprotein; lipoprotein lipase; enzymes which catalyze a series of reactions leading to the generation of hydrogen peroxide or a reduced coenzyme from glycerol; and an enzyme which catalyzes a reaction leading to the conversion of hydrogen peroxide or a reduced coenzyme into another substance, and
a second reagent that comprises a second selective reaction promoter, which is capable of reacting lipoprotein lipase selectively with triglycerides contained in very low density lipoprotein, intermediate density lipoprotein, low density lipoprotein and high density lipoprotein.

14. The reagent according to claim 13, wherein the first reagent and/or the second reagent further comprises a substance which is involved in a reaction leading to the derivation of some signal from hydrogen peroxide or a reduced coenzyme.

15. The reagent according to claim 13, wherein the second selective reaction promoter is an ether or ester compound of a polyoxyalkylene.

16. The reagent according to claim 15, wherein m/n ratio is in the range of 1.1 to 1.2, where m is the average mole number of the added polyoxyalkylene in its ether or ester compound which is used as the first selective reaction promoter and n is the average mole number of the added polyoxyalkylene in its ether or ester compound which is used as the second selective reaction promoter.

17. The reagent according to claim 16, wherein m is in the range of 7.7 to 18 and n is in the range of 7 to 15.

18. The reagent according to claim 16, wherein m is in the range of 11 to 12 and n is 10.

19. The reagent according to claim 13, wherein the ether or ester compound of a polyoxyalkylene used as the first selective reaction promoter is at least one selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

20. The reagent according to claim 13, wherein the second selective reaction promoter is at least one ether or ester compound of a polyoxyalkylene selected from the group consisting of polyoxyalkylene straight-chain alkyl ethers, polyoxyalkylene branched-chain alkyl ethers, polyoxyalkylene straight-chain alkylphenyl ethers, polyoxyalkylene branched-chain alkylphenyl ethers, polyoxyalkylene straight-chain fatty acid esters, polyoxyalkylene branched-chain fatty acid esters, polyoxyalkylene straight-chain alkyl substituted benzoic acid esters and polyoxyalkylene branched-chain alkyl substituted benzoic acid esters.

21. The reagent according to claim 13, wherein the polyoxyalkylene is polyoxyethylene.

22. The reagent according to claim 13, wherein the first selective reaction promoter is polyoxyethylene nonylphenyl ether in which the average mole number of added polyoxyethylene m is in the range of 11 to 12 and the second selective reaction promoter is polyoxyethylene nonylphenyl ether in which the average mole number of added polyoxyethylene n is 10.

23. The reagent according to claim 13, wherein the first reagent and/or the second reagent further comprises a reaction assistant.

24. The reagent according to claim 23, wherein the reaction assistant is a polysaccharide or derivative thereof, a polyanion, a halogen ion, a metal ion, or lectin.

25. The reagent according to claim 13, wherein the activity of the lipoprotein lipase contained in the first reagent depends on the concentration of a surfactant, while that of the lipoprotein lipase contained in the second reagent hardly depends on the concentration of a surfactant.
